(19) United States
(12) Patent Application Publication
(10) Pub. No.: US 2005/0021240 A1
(43) Pub. Date: Jan. 27, 2005

(54) Systems, methods and computer program products for guiding selection of a therapeutic treatment regimen based on the methylation status of the DNA

(75) Inventors: Kurt Berlin, Stahnsdorf (DE); Alexander Olek, Berlin (DE); Christian Piepenbrock, Berlin (DE)

Correspondence Address: DAVIS WRIGHT TREMAINE, LLP, 2600 CENTURY SQUARE, 1501 FOURTH AVENUE, SEATTLE, WA, 98101-1688, US

(73) Assignee: Epigenomics AG, Berlin (DE)

(21) Appl. No.: 10/857,105

(22) Filed: May 28, 2004

Related U.S. Application Data
(63) Continuation-in-part of application No. 10/774,052, filed on Feb. 6, 2004.
Application No. 10/774,052 is a continuation-in-part of application No. 09/705,302, filed on Nov. 2, 2000, now abandoned.

(51) Int. Cl. G06F 17/60; C12Q 1/68; G06F 19/00; G01N 33/48; G01N 33/50

(52) U.S. Cl. 702/20; 705/3

(57) ABSTRACT

Systems, methods and computer program products for guiding selection of a therapeutic treatment regimen or a preventive therapeutic treatment regimen are disclosed. The method comprises (A) providing to a computing device comprising a first knowledge base comprising information about a plurality of different methylation statuses at selected sites of the DNA in cells with a known disease or medical condition and/or healthy cells, a second knowledge base comprising a plurality of expert rules for evaluating and selecting a type of disease or medical condition based on the methylation status at selected sites of the DNA of a patient, (B) generating in said computing device a ranked listing of diseases or medical conditions based on the information about the methylation status at selected sites of the DNA of the patient, the first knowledge base and the second knowledge base.

### CROSS-REFERENCE TO RELATED APPLICATIONS

This application is a continuation-in-part application of co-pending application U.S. Ser. No. (to be assigned), filed Feb. 6, 2004, which is a continuation-in-part application of application U.S. Ser. No. 09/705,302, filed Nov. 11, 2000, now abandoned. The priority benefit of all earlier applications is claimed in accordance with 35 U.S.C. § 120, and all references as cited herein are incorporated in their entirety for the purposes of the present invention.

### TECHNICAL FIELD OF THE INVENTION

This invention concerns systems, methods and computer program products for guiding the selection of therapeutic treatment regimens for complex disorders such as cancer viral and/or bacterial infection, wherein a ranking of available treatment regimens is generated based on information about the methylation status at selected sites of the DNA of the patient and advisory information clinically useful for treating patients is provided.

### BACKGROUND OF THE INVENTION

### (DESCRIPTION OF THE RELATED ART)

The levels of observation that have been well studied by the methodological developments of recent years in molecular biology include the gene itself, the translation of genes in RNA, and the resulting proteins. When, during the course of the development of an individual, a gene is switched on, and how the activation and inhibition of certain genes in certain cells and tissues is controlled, can be correlated with a high degree of probability with the extent and the character of the methylation of the gene or the genome. In this regard, it is reasonable to assume that pathogenic conditions are expressed in a modified methylation pattern of individual genes or of the genome.

The state of the art is a method which allows the study of the methylation pattern of individual genes. More recent additional developments of this method also allow the analysis of minute quantities of starting material, where, however, the total number of measurement points remains at most a two-digit number, in theoretical range of values of at east 107 measurement points.

1. State of the Art of Molecular Analysis of Cell Phenotypes

The study of gene expression can be at the RNA level or at the protein level. Both levels in principle reflect important phenotypic parameters. Protein assays using two-dimensional gels (McFarrel method) have been known for approximately 15 years. Using these assays, it is possible to elaborate the analysis of the chromatographic positions of several thousand proteins. Very early on, such electropherograms were already processed or evaluated with data processing means. In principle, the validity of the method is high, however, it is inferior to the modern methods of gene expression based on RNA analysis in two regards.

In particular, the detection of proteins that are of regulatory importance, from small quantities of cells, fails because of the fact that the sensitivity of the methods used is much too low. Indeed, in contrast to nucleic acids, proteins cannot be amplified. In addition, the method is very complex, not amenable to automation, and very expensive. In contrast, RNA analysis presents considerable advantages, and due to of the use of PCR it is more sensitive. Above all, each RNA species recognized to be important can be identified immediately by its sequence.

Overexpression or underexpression of individual RNAs with a known sequence can usually be easily detected; however, in connection with the applications discussed here, they are only valid in exceptional cases.

The method of “differential displays” at best allows a semiquantitative study of expression. Expression products amplified by PCR are separated by gel e lectrophoresis. The validity is limited as a result of the resolution of the gel electrophoresis. In addition, the method is insufficiently sensitive and robust for use in routine diagnosis (Liang, P. and Pardee, A. B., Science 257, 967-971).

Genes with high overexpression or underexpression are frequently identified by subtractive techniques. Here, cDNA clones of a cell or tissue species to be examined are plated. Against the clones, cDNA is hybridized as comparison material. Expression patterns cannot be reliably prepared using this technique.

One activity of the American “human genome project” is the systematic sequencing of expressed genes. The data obtained from this can be used to build expression chips, which allow the study of practically all expressed sequences of a cell or tissue type in a single experiment.

2. State of the Art in the Analysis of Cancer Diseases

Mutations in genes always trigger cancer diseases, that is, cell degeneration. The causes of these mutations can be exogenous influences, or events in the cell. In a few exceptional cases, an individual mutation, which frequently affects larger regions of the genome (translocations, deletions), results in the degeneration of the cell; but in most cases a chain of mutations on different genes is involved, and it is only their combined effect that results in the malignant disease. These results on the DNA level are also reflected on the RNA and protein levels. In this context, it is highly probable that a multiplication occurs, because it is certain that in many cases the quantity and type of one RNA influences the extent of the synthesis of several other RNA species. This leads to a change in the synthesis rates of the corresponding proteins, which, in turn, can result in deregulating metabolism, and thus initiate the mechanism of regulation and counter regulation. The result is a gene expression pattern of the cells in question, that has been modified in a very specific (but largely non determinable) manner, the specificity is for a certain carcinoma, for the stage of the carcinoma, and the degree of malignancy of the carcinoma. So far, such phenomena have been outside the realm of study of natural sciences. Indeed, it has been impossible to examine the gene expression or the metabolism of a cell in its totality. Chip technology for the first time provided such a possibility (Schena, M. et al., Science 270, 467-470).

If one wishes to solve the diagnostic problem of early diagnosis of tumors on the molecular level, then one is confronted, today, with an insurmountable difficulty, with very few exceptions: Because, for most tumors, the knowledge of the molecular events, that is, the different mutations, is only fragmentary; researchers do not know what to look for in medical examination material. This means it is absolutely impossible to apply the remarkable sensitivity and specificity of the polymerase chain reaction. Examples are certain intestinal tumors, Ewing's sarcoma, and certain forms of leukemia, which are in fact each defined by a single, precisely described mutation. In those cases, it is possible to identify the degenerated cell among millions of normal cells. However, even within these apparently unambiguously defined tumor groups, there are such differences in the behavior that the conclusion must be drawn that additional unknown genetic parameters (such as, for example, the genetic background of the individual) play an important role. Immunological tumor markers are helpful auxiliary parameters, but they continue to make only a modest contribution, in addition to the other conventional diagnostic parameters. However, they can be used for the purpose of preselecting suspect cells.

Histology plays an important and indispensable role in the identification of degenerated tissues, but not precisely in early diagnosis.

Thus, because most tumors are not sufficiently characterized for diagnostic purposes on the molecular level, as a rule, no possibilities exist to proceed to a subdivision into stages or even a subdivision by degrees of risk. Such a subdivision, however, is an absolute prerequisite for an improved selection of treatments and, above all, for the development of effective new drugs and of gene therapy.

3. State of the Art in Research on the Number, Type and Properties of the Possible Stable States of Cells of Higher Organisms

In recent times, there has been an increase in the number of indications that complex regulatory systems (an excellent example of which is cell regulation), when left alone, can exist in only a limited number of stable states, above a critical minimum complexity and below a critical maximum connectivity (of the average number of the components, with which any given component is connected) (Kauffman, S. A., Origins of Order, Oxford University Press, 1993). In this context, the word state should be understood as the concept of selection for the general phenomenon. In connection with cells as biological regulatory systems, one can also talk of differentiation state or cell type. Although no such connection has been demonstrated—and even a mere 1 imitation of the possible states for biological systems has not been demonstrated—the practical implications would be of very great importance: If, regarding the constant information content of the cells of an organism (de facto, such constancy essentially exists within one species), there were only a limited number of stable states, then it would be likely that degenerated cells could also be in only one of these states or in a transition between the possible states. At this time, there is no possibility to define these states on a molecular basis. It is hardly possible to achieve a correlation between the individual states and the behavior of the cells according to the state of the art. However, such an analysis could make decisive contributions to the diagnosis and prognosis of diseases. It is even possible that a correlation could be established between the possible states of diseased cells and the best suited therapy. Furthermore, it is probable that such a method could also have a decisive influence in the selection of the time of treatment. For example, if one were to discover that the cells of a tumor are in a transition between possible states, one could assume that such a population of cells would be more likely to yield to the selection pressure resulting from the treatment, and thus could escape more easily. A cell population in such a scenario, within such transitional states, would have a considerably increased flexibility, and it would be easily forced into a possible stable state, in which the selection pressure would be eliminated, and the treatment would thus be without effect. A method which could classify cells and cell groups according to states would then also contribute to recognizing, understanding and possibly solving such problems. However, according to the state of the art, it is not possible to determine whether only a limited number of states of cells exists. It follows that it is not possible to differentiate groups of cells according to an abstract criterion concerning their states, and to predict these states with a certain behavior of the cells.

4. Hereditary Diseases

Today, the genetic map of the human genome comprises 2500 so-called microsatellites. These instruments are used to locate a multitude of genes, usually genes whose defect causes a genetic disease, per linkage analysis, and then to identify them. Common genetic diseases caused by a single defective gene are thus elucidated, from the point of view of the geneticist's principle, polygenic diseases should also be understood in this manner. Many polygenic diseases are very common, so common that they are included among the so-called wide-spread diseases. Asthma and diabetes are examples. Many carcinoma types are also included. The use of the above-described strategy of linkage analysis also produced enormous initial successes. In many instances, numerous causal genes of important polygenic diseases such as diabetes, schizophrenia, atherosclerosis and obesity have been found. Besides the availability of the molecular biology laboratory techniques proper, the availability of a relatively large number of patients and relatives affected by each disease is a crucial prerequisite for genetic elucidation. In the past two years it has become apparent that the number of several hundred patients that were originally used for the linkage analysis of polygenic diseases very likely is too low by one order of magnitude. This applies, in any case, to cases where the entire spectrum of the causal gene is to be elucidated. Because the level of manual work required for such a linkage analysis is extraordinarily high, only very slow progress can be expected in the analysis of polygenic diseases. Alternative strategies are sought because it is precisely these diseases that are of enormous social and economic importance.

5. State of the Art in Methylation Analysis

The modification of the genomic base cytosine to 5′-methylcytosine represents the epigenetic parameter which to date is the most important one and has been best examined. Nevertheless, methods exist today to determine comprehensive genotypes of cells and individuals, but no comparable methods exist to date to generate and evaluate epigenotypic information on a large scale.

In principle, there are three methods that differ in principle for determining the 5-methyl state of a cytosine in the sequence context.

The first method is based in principle on the use of restriction endonucleases (RE), which are methylation-sensitive“. REs are characterized in that they produce a cut in the DNA at a certain DNA sequence which is usually 4-8 bases long. The position of such cuts can be detected by gel electrophoresis, transfer to a membrane and hybridization. Methylation-sensitive means that certain bases within the recognition sequence must be unmethylated for the step to occur. The band pattern after a restriction cut and gel electrophoresis thus changes depending on the methylation pattern of the DNA. However, most CpG that can be methylated are outside of the recognition sequences of REs, and thus cannot be examined.

The sensitivity of this method is extremely low (Bird, A. P., Southern, E. M., J. Mol. Biol. 118, 27-47). A variant combines PCR with this method; an amplification by two primers located on both sides of the recognition sequence occurs after a cut only if the recognition sequence is in the methylated form. In this case, the sensitivity theoretically increases to a single molecule of the target sequence; however, only individual positions can be examined, at great cost (Shemer, R. et al., PNAS 93, 6371-6376).

The second variant is based on the partial chemical cleavage of whole DNA, using the model of a Maxam-Gilbert sequencing reaction, ligation of adaptors to the ends thus generated, amplification with generic primers, and separation by gel electrophoresis. Using this method, defined regions having a size of less than thousands of base pairs can be examined. However, the method is so complicated and unreliable that it is practically no longer used (Ward, C, et al., J. Biol. Chem. 265, 3030-3033).

A new method for the examination of DNA to determine the presence of 5-methylcytosine is based on the specific reaction of bisulfite with cytosine. The latter is converted under appropriate conditions into uracil, which, as far as base pairing is concerned, is equivalent to thymidine, and which also corresponds to another base. 5-Methylcytosine is not modified. As a result, the original DNA is converted in such a manner that methylcytosine, which originally could not be distinguished from cytosine by its hybridization behavior, now can be detected by “normal” molecular biological techniques. All of these techniques are based on base pairing, which can now be completely exploited. The state of the art, as far as sensitivity is concerned, is defined by a method which includes the DNA to be examined in an agarose matrix, intended to prevent the diffusion and renaturing of the DNA (bisulfite reacts only with single-stranded DNA) and to replace all precipitation and purification steps by rapid dialysis (Olek, A., et al., Nucl. Acids. Res. 24, 5064-5066). Using this method, individual cells can be examined, which illustrates the potential of the method. However, so far only individual regions up to approximately 3000 base pairs in length have been examined, and an overall examination of cells to identify thousands of possible methylation events is not possible. However, this method is not capable of reliably analyzing minute fragments from small sample quantities. In spite of protection against diffusion, such samples are lost through the matrix.

6. State of the Art in the use of the Bisulfite Technique

To date, barring few exceptions, (for example, Zeschnigk, M. et al., Eur. J. Hum. Gen. 5, 94-98; Kubota, T. et al., Nat. Genet. 16, 16-17), the bisulfite technique is only used in research. However, short specific pieces of a known gene after bisulfite treatment are routinely amplified and either completely sequenced (Olek, A. and Walter, J., Nat. Genet. 17, 275-276) or the presence of individual cytosine positions is detected by a “primer extension reaction” (Gonzalgo, M. L. and Jones, P. A., Nucl. Acids. Res. 25, 2529-2531), or enzyme cut (Xiong, Z. and Laird, P. W., Nucl. Acids. Res. 25, 2532-2534). All these references are from the year 1997. The concept of using complex methylation patterns for correlation with phenotypic data pertaining to complex genetic diseases, much less via an evaluation algorithm such as, for example, a neural network, has, so far, gone unmentioned in the literature; moreover, it cannot be performed according to the methodologies of the state of the art.

7. State of the Art with Respect to Methylation and the diagnosis of Human Diseases

In the past, modification of the methylation pattern was analyzed in order to study and understand the genetic mechanisms which are involved in the outbreak or the progression of a disease. All this research was done in a piece-by-piece fashion by studying only one gene/chromosomal region at a time and no diagnosis/therapeutic treatment regimen was based on the methylation pattern modifications. In fact, the type of disease associated with the modification of the methylation pattern was known before methylation analysis was performed. Therefore, the following publications only indicate the widespread connection between modifications of the methylation patterns and human diseases. Modifications can include both hyper- or hypomethylation of selected sites of the DNA.

Diseases that are associated with a modification of the methylation patterns are, for example:
Leukemia (Aoki E et al. “Methylation status of the p15INK4B gene in hematopoietic progenitors and peripheral blood cells in myelodysplastic syndromes” Leukemia 2000 April;14(4):586-93; Nosaka K et al. “Increasing methylation of the CDKN2A gene is associated with the progression of adult T-cell leukemia” Cancer Res 2000 Feb. 15;60(4):1043-8;

Asimakopoulos F A et al. “ABL1 methylation is a distinct molecular event associated with clonal evolution of chronic myeloid leukemia” Blood 1999 Oct. 1;94(7):2452-60; Fajkusova L. et al. “Detailed Mapping of Methylcytosine Positions at the CpG Island Surrounding the Pa Promoter at the bcr-abl Locus in CML Patients and in Two Cell Lines, K562 and BV173” Blood Cells Mol Dis 2000 June;26(3):193-204; Litz C E et al. “Methylation status of the major breakpoint cluster region in Philadelphia chromosome negative leukemias” Leukemia 1992 January;6(1):35-41);
Head and neck cancer (Sanchez-Cespedes M et al. “Gene promoter hypermethylation in tumors and serum of head and neck cancer patients” Cancer Res 2000 Feb. 15;60(4):892-5);
Hodgkin's disease (Garcia JF et al. “Loss of p16 protein expression associated with methylation of the p16INK4A gene is a frequent finding in Hodgkin's disease” Lab Invest 1999 December;79(12):1453-9);
Gastric cancer (Yanagisawa Y et al. “Methylation of the hMLH1 promoter in familial gastric cancer with microsatellite instability” Int J Cancer 2000 Jan. 1;85(1):50-3);
Prostate cancer (Rennie P S et al. “Epigenetic mechanisms for progression of prostate cancer” Cancer Metastasis Rev 1998-99; 17(4):401-9);
Renal cancer (Clifford S C et al. “Inactivation of the von Hippel-Lindau (VHL) tumor suppressor gene and allelic losses at chromosome arm 3p in primary renal cell carcinoma: evidence for a VHL-independent pathway in clear cell renal tumourigenesis” Genes Chromosomes Cancer 1998 July;22(3):200-9);
Bladder cancer (Sardi I et al. “Molecular genetic alterations of c-myc oncogene in superficial and locally advanced bladder cancer” Eur Urol 1998;33(4):424-30);
Breast cancer (Mancini D N et al. “CpG methylation within the 5′ regulatory region of the BRCA1 gene is tumor specific and includes a putative CREB binding site” Oncogene 1998 Mar. 5;16(9):1161-9; Zrihan-Licht S et al. “DNA methylation status of the MUC1 gene coding for a breast-cancer-associated protein” Int J Cancer 1995 Jul. 28;62(3):245-51; Kass D H et al. “Examination of DNA methylation of chromosomal hot spots associated with breast cancer” Anticancer Res 1993 September-October; 13(5A): 1245-51);
Burkitt's lymphoma (Tao Q e t al. “Epstein-Barr virus (EBV) in endemic Burkitt's lymphoma: molecular analysis of primary tumor tissue” Blood 1998 Feb. 15;91(4):1373-81)
Wilms tumor (Kleymenova E V et al. “Identification of a tumor-specific methylation site in the Wilms tumor suppressor gene” Oncogene 1998 Feb. 12;16(6):713-20);
Prader-Willi/Angelman syndrome (Zeschnigh et al. “Imprinted segments in the human genome: different DNA methylation patterns in the Prader-Willi/Angelman syndrome region as determined by the genomic sequencing method” Human Mol. Genetics (1997) (6)3 pp 387-395; Fang P et al. “The spectrum of mutations in UBE3A causing Angelman syndrome” Hum Mol Genet 1999 January;8(l):129-35);
ICF syndrome (Tuck-Muller et al. “CMDNA hypomethylation and unusual chromosome instability in cell lines from ICF syndrome patients” Cytogenet Cell Genet 2000;89(1-2):121-8);
Dermatofibroma (Chen T C et al. “Dermatofibroma is a clonal proliferative disease” J Cutan Pathol 2000 January;27(1):36-9);
Hypertension (Lee S D et al. “Monoclonal endothelial cell proliferation is present in primary but not secondary pulmonary hypertension” J Clin Invest 1998 Mar. 1; 101(5):927-34);
Pediatric Neurobiology (Campos-Castello J et al. “The phenomenon of genomic “imprinting” and its implications in clinical neuropediatrics” Rev Neurol 1999 Jan. 1-15;28(1):69-73);
Autism (Klauck S M et al. “Molecular genetic analysis of the FMR-1 gene in a large collection of autistic patients” Hum Genet 1997 August; 100(2):224-9);
Ulcerative colitis (Gloria L et al. “DNA hypomethylation and proliferative activity are increased in the rectal mucosa of patients with long-standing ulcerative colitis” Cancer 1996 Dec. 1;78(11):2300-6);
Fragile X syndrome (Hornstra I K et al. “High resolution methylation analysis of the FMR1 gene trinucleotide repeat region in fragile X syndrome” Hum Mol Genet 1993 October;2(10):1659-65); and
Huntington's disease (Ferluga J et al. “Possible organ and age-related epigenetic factors in Huntington's disease and colorectal carcinoma” Med Hypotheses 1989 May;29(1):51-4).

The above listing does only give a brief overview of the current status of diseases that have been linked to modified methylation patterns of certain genes (e.g., oncogenes) and/or their regulatory regions, such as the promoter sequences thereof. In addition, the methylation pattern of certain genes has been used for a distinction between different subtypes of certain cancer diseases, such a s leukemia subtypes. Additional diseases and/or disease states that have been linked to modified methylation patterns of certain genes (e.g. oncogenes) and/or their regulatory regions, such as the promoter sequences thereof are also, for example, reviewed in the following publications and the references as cited therein: Keku T O, Rakhra-Burris T, Millikan R. Gene testing: what the health professional needs to know. J Nutr. 2003 November;133(11 Suppl 1):3754S-3757S; Loktionov A. Common gene polymorphisms and nutrition: emerging links with pathogenesis of multifactorial chronic diseases. J Nutr Biochem. 2003 August;14(8):426-51; Jichlinski P. New diagnostic strategies in the detection and staging of bladder cancer. Curr Opin Urol. 2003 September;13(5):351-5. Mason J B. Biomarkers of nutrient exposure and status in one-carbon (methyl) metabolism. J Nutr. 2003 March;133 Suppl 3:941S-947S; Lievers K J, Kluijtmans L A, Blom H J. Genetics of hyperhomocysteinaemia in cardiovascular disease. Ann Clin Biochem. 2003 January;40(Pt 1):46-59; Novik K L, Nimmrich I, Genc B, Maier S, Piepenbrock C, Olek A, Beck S. Epigenomics: genome-wide study of methylation phenomena. Curr Issues Mol Biol. 2002 October;4(4):111-28. Dong C, Yoon W, Goldschmidt-Clermont P J. DNA methylation and atherosclerosis. J Nutr. 2002 August;132(8 Suppl):2406S-2409S; Lehmann U, Kreipe H. Real-time PCR analysis of DNA and RNA extracted from formalin-fixed and paraffin-embedded biopsies. Methods. 2001 December;25(4):409-18; Wong I H, Lo Y M, Johnson P J. Epigenetic tumor markers in plasma and serum: biology and applications to molecular diagnosis and disease monitoring. Ann NY Acad Sci. 2001 September;945:36-50; Jubb A M, Bell S M, Quirke P. Methylation and colorectal cancer. J Pathol. 2001 September;195(1):111-34; Richer L P, Shevell M I, Miller S P. Diagnostic profile of neonatal hypotonia: an 11-year study. Pediatr Neurol. 2001 July;25(l):32-7; Lee M E, Wang H. Homocysteine and hypomethylation. A novel link to vascular disease. Trends Cardiovasc Med. 1999 January-February;9(1-2):49-54; Hoffmann G F, Surtees R A, Wevers R A. Cerebrospinal fluid investigations for neurometabolic disorders. Neuropediatrics. 1998 April;29(2):59-71; Holliday R, Grigg G W. DNA methylation and mutation. Mutat Res. 1993 January;285(1):61-7; Cooper D N, Krawczak M. The mutational spectrum of single base-pair substitutions causing human genetic disease: patterns and predictions. Hum Genet. 1990 June;85(1):55-74; Fackler M J, McVeigh M, Evron E, Garrett E, Mehrotra J, Polyak K, Sukumar S, Argani P. DNA methylation of RASSF1A, HIN-1, RAR-beta, Cyclin D2 and Twist in in situ and invasive lobular breast carcinoma. Int J Cancer. 2003 Dec. 20;107(6):970-5; Pradhan S, Esteve P O. Mammalian DNA (cytosine-5) methyltransferases and their expression. Clin Immunol. 2003 October;109(1):6-16; Hamet P, Tremblay J. Genes of aging. Metabolism. 2003 October;52(10 Suppl 2):5-9; Coleman W B. Mechanisms of human hepatocarcinogenesis. Curr Mol Med. 2003 September;3(6):573-88; Cleary J D, Pearson C E. The contribution of cis-elements to disease-associated repeat instability: clinical and experimental evidence. Cytogenet Genome Res. 2003;100(1-4):25-55; Li S, Hursting S D, Davis B J, McLachlan J A, Barrett J C. Environmental exposure, DNA methylation, and gene regulation: lessons from diethylstilbesterol-induced cancers. Ann NY Acad Sci. 2003 March;983:161-9. Muegge K, Young H, Ruscetti F, Mikovits J. Epigenetic control during lymphoid development and immune responses: aberrant regulation, viruses, and cancer. Ann NY Acad Sci. 2003 March;983:55-70; Schagdarsurengin U, Wilkens L, Steinemann D, Flemming P, Kreipe H H, Pfeifer G P, Schlegelberger B, Dammann R. Frequent epigenetic inactivation of the RASSF1A gene in hepatocellular carcinoma. ]Oncogene. 2003 Mar. 27;22(12): 1866-71; Sekigawa I, Okada M, Ogasawara H, Kaneko H, Hishikawa T, Hashimoto H. DNA methylation in systemic lupus erythematosus. Lupus. 2003;12(2):79-85; Jaenisch R, Bird A. Epigenetic regulation of gene expression: how the genome integrates intrinsic and environmental signals. Nat Genet. 2003 March;33 Suppl:245-54; Harden S V, Guo Z, Epstein J I, Sidransky D. Quantitative GSTP1 methylation clearly distinguishes benign prostatic tissue and limited prostate adenocarcinoma. J Urol. 2003 March;169(3):113842. Chen W Y, Zeng X, Carter M G, Morrell C N, Chiu Yen R W, Esteller M, Watkins D N, Herman J G, Mankowski J L, Baylin S B. Heterozygous disruption of Hic1 predisposes mice to a gender-dependent spectrum of malignant tumors. Nat Genet. 2003 February;33(2):197-202. Epub 2003 Jan. 21; Arenas-Huertero F, Recillas-Targa F. Chromatin epigenetic modifications in cancer generation Gac Med Mex. 2002 November-December;138(6):547-55; Pelham J T, Irwin P J, Kay P H. Genomic hypomethylation in neoplastic cells from dogs with malignant lymphoproliferative disorders. Res Vet Sci. 2003 February;74(l):1014, Brooks W H. Systemic lupus erythematosus and related autoimmune diseases are antigen-driven, epigenetic diseases. Med Hypotheses. 2002 December;59(6):73641; Novik K L, Nimmrich I, Genc B, Maier S, Piepenbrock C, Olek A, Beck S. Epigenomics: genome-wide study of m ethylation phenomena. Curr Issues Mol Biol. 2002 October;4(4): 111-28; Nazarenko S A. Impaired epigenetic gene activity regulation and human diseases Vestn Ross Akad Med Nauk. 2001;(10):43-8; Li E. Chromatin modification and epigenetic reprogramming in mammalian development. Nat Rev Genet. 2002 September;3(9):662-73; Dong C, Yoon W, Goldschmidt-Clermont P J. DNA methylation and atherosclerosis. J Nutr. 2002 August;132(8 Suppl):2406S-2409S; Issa J P. Epigenetic variation and human disease. J Nutr. 2002 August;132(8 Suppl):2388S-2392S; James S J, Melnyk S, Pogribna M, Pogribny I P, Caudill M A. Elevation in S-adenosylhomocysteine and DNA hypomethylation: potential epigenetic mechanism for homocysteine-related pathology. J Nutr. 2002 August;132(8 Suppl):2361S-2366S. Robertson K D. DNA methylation and chromatin—unraveling the tangled web. Onco-gene. 2002 Aug. 12;21(35):5361-79; Fruhwald M C, Plass C. Global and gene-specific methylation patterns in cancer: aspects of tumor biology and clinical potential. Mol Genet Metab. 2002 January;75(l): 1-16; Wong I H, Lo Y M, Johnson P J. Epigenetic tumor markers in plasma and serum: biology and applications to molecular diagnosis and disease monitoring. Ann NY Acad Sci. 2001 September;945:36-50; Van Seuningen I, Pigny P, Perrais M, Porchet N, Aubert J P. Transcriptional regulation of the 11p 15 mucin genes. Towards new biological tools in human therapy, in inflammatory diseases and cancer? Front Biosci. 2001 Oct. 1;6:D1216-34; Jubb A M, Bell S M, Quirke P. Methylation and colorectal cancer. J Pathol. 2001 September;195(1):111-34; Urnov F D, Wolffe A P. Above and within the genome: epigenetics past and present. J Mammary Gland Biol Neoplasia. 2001 April;6(2): 153-67; Jones P A, Takai D. The role of DNA methylation in mammalian epigenetics. Science. 2001 Aug. 10;293(5532):1068-70; Maegawa S, Yoshioka H, Itaba N, Kubota N, Nishihara S, Shirayoshi Y, Nanba E, Oshimura M. Epigenetic silencing of PEG3 gene expression in human glioma cell lines. Mol Carcinog. 2001 May;31(1):1-9; Rao A, Avni O. Molecular aspects of T-cell differentiation. Br Med Bull. 2000;56(4):969-84; Rakyan V K, Preis J, Morgan H D, Whitelaw E. The marks, mechanisms and memory of epigenetic states in mammals. Biochem J. 2001 May 15;356(Pt 1):1-10; Robertson K D, Wolffe A P. DNA methylation in health and disease. Nat Rev Genet. 2000 October;1(1): 11-9; El-Osta A, Wolffe A P. DNA methylation and histone deacetylation in the control of gene expression: basic biochemistry to human development and disease. Gene Expr. 2000;9(1-2):63-75; Jirtle R L, Sander M, Barrett J C. Genomic imprinting and environmental disease susceptibility. Environ Health Perspect. 2000 March;108(3):271-8; Wolffe A P, Matzke M A; Epigenetics: regulation through repression. Science. 1999 Oct. 15;286(5439):481-6; Schmutte C, Jones P A. Involvement of DNA methylation in human carcinogenesis. Biol Chem. 1998 April-May;379(4-5):377-88; Tycko B. DNA methylation in genomic imprinting. Mutat Res. 1997 April;386(2):13140; Nakao M, Sasaki H. Genomic imprinting: significance in development and diseases and the molecular mechanisms. J Biochem (Tokyo). 1996 September;120(3):467-73; Yung R L, Johnson K J, Richardson B C. New concepts in the pathogenesis of drug-induced lupus. Lab Invest. 1995 December;73(6):746-59; Guala A, Lerone M, Cirillo Silengo M. Genomic imprinting and human pathology. I. General Part] Pediatr Med Chir. 1995 July-August;17(4):311-21; Holliday R. Epigenetic inheritance based on DNA methylation. EXS. 1993;64:452-68; Driscoll D J, Waters M F, Williams C A, Zori R T, Glenn C C, Avidano K M, Nicholls R D. A DNA methylation imprint, determined by the sex of the parent, distinguishes the Angelman and Prader-Willi syndromes. Genomics. 1992 August;13(4):917-24; and Holliday R. The inheritance of epigenetic defects. Science. 1987 Oct. 9;238(4824):163-70.

All the above-cited documents are hereby incorporated by reference for the purposes of the present invention.

8. Personalized Medicine

A successful therapeutic treatment of a patient in need of such a treatment generally depends on several factors.

First, a reliable diagnosis of the disease or the medical condition has to be achieved. In case of infectious diseases, cancer or other acute life-threatening diseases, this diagnosis has to be fast and efficient, since time plays a crucial role in the survival rate of patients suffering from those diseases. The ideal diagnosis would therefore rely on data of the patient which is easy to assess and does not involve a time-consuming diagnosis procedure. In addition, one would prefer the least invasive way in order to achieve samples from a patient to be examined. One aspect of the methods to be patented here provides new possibilities for the differential diagnosis of, for example, cancer diseases.

Second, a therapeutic treatment of an individual patient becomes more effective if the diagnosis is precise. Currently, for example cancer is sometimes treated with a standard “cocktail” of anti-cancer drugs exhibiting severe side effects for the patient. Nevertheless, the survival rate of at least some types of cancer is low. Once the type of cancer (or other disease) is precisely determined, an individual treatment regime for this type of disease would be exponentially more effective than any other treatment regimen. Effectiveness in this case depends directly on the individual application of the therapeutic treatment to the patient. An even more effective treatment would be possible, if the treatment regimen would be cross-checked with regimens already successfully applied to other patients.

Further, a precise diagnosis of the disease would lead to reduced costs for the individual treatment regimen, since unnecessary and ineffective medication is avoided.

Further, therapeutic treatment regimens for human diseases, such as AIDS and cancer are increasingly complex. New data and new therapeutic treatment regimens continue to modify the treatments available, and it is difficult for all but the specialist to remain current on the latest treatment information.

Further, even those who are current on the latest treatment information require time to assimilate that information and understand how it relates to other treatment information in order to provide the best available treatment for a patient. Combination therapeutic treatment regimens exacerbate this problem by making potential drug interactions even more complex.

Finally, an increasingly sophisticated patient population, in the face of a vast volume of consumer information on the treatment of disease, makes the mere statement of a treatment regime, without explanation, difficult for the patient to accept.

Another desirable form of treatment would comprise a preventive kind of treatment regimen which could be applied at the earliest stage of an upcoming disease. In order to know when to apply such a treatment regimen, one would need a form of diagnosis that could determine changes in the health status of the patient even before an outbreak of an acute disease could be diagnosed. This outbreak could then be prevented or reduced in severity by applying a preventive treatment regimen to such non-acute patient.

Taken together, the ideal treatment regimen would combine all the above-mentioned factors in order to apply the most effective medication to the individual patient. This individual diagnosis/medication regimen can be summarized using the term “personalized medicine”.

U.S. Pat. No. 5,672,154 to Sillen describes a method for giving patients individualized, situation-dependent medication advice. The recommended type of medicine may include at least two different medicines. No means for ranking multiple treatment options is disclosed, and no means for explaining why treatment options were rejected is given. Rather, this system is primarily concerned with generating new rules from patient information to optimize a particular therapy for diseases such as Parkinson's disease, epilepsy and abnormal blood pressure. Sillen does not disclose the need for a more precise diagnosis or the use of DNA-methylation for the individualized medication advice.

U.S. Pat. No. 5,918,568 to Gjerlov describes a method of medicating and individualizing treatment shampoo for dermatological disturbances of companion animals. Gjerlov further describes a system for customized provision of medicated shampoos which are individualized for treatment of specific dermatological disturbances of specific and individual companion animals. The method disclosed involves diagnosing the dermatological disturbance and then adding to a pre-mixed base shampoo a pre-mixed, medically effective amount of concentrate correlated to the particular dermatological disturbance, and then the composition is provided to the owner of the animal. Further, a kit apparatus to carry out the method and packaging for shipment and display of the apparatus is disclosed.

U.S. Pat. No. 5,694,950 to McMichael describes a method and system for use in treating a patient with immunosuppressants such as cyclosporin. An expert system is employed to generate a recommendation on whether the immunosuppressant dosage should be changed and, if so, how. Ranking or selection among a plurality of different combination therapeutic treatment regimens is not suggested.

U.S. Pat. No. 5,594,638 to Iliff describes a medical diagnostic system that provides medical advice to the general public over a telephone network. This system is not concerned with generating a recommendation for a combination therapeutic treatment regimen for a known disease (see also U.S. Pat. No. 5,660,176 to Iliff).

U.S. Pat. No. 6,081,786 to Barry et al. describes systems, methods and computer program products for guiding selection of a therapeutic treatment regimen for a known disease such as HIV infection are disclosed. The method comprises (a) providing patient information to a computing device (the computer device comprising: a first knowledge base comprising a plurality of different therapeutic treatment regimens for the disease; a second knowledge base comprising a plurality of expert rules for selecting a therapeutic treatment regimen for the disease; and a third knowledge base comprising advisory information useful for the treatment of a patient with different constituents of the different therapeutic treatment regimens; and (b) generating in the computing device a listing (preferably a ranked listing) of therapeutic treatment regimens for the patient; and (c) generating in the computing device advisory information for one or more treatment regimens in the listing based on the patient information and the expert rules.

EP 1176539 to Schapiro et al. discloses a method for effecting computer-implemented decision-support in the selection of a drug therapy of patients having a viral disease. A rules database is provided and patient data is entered including genotype data on the viral genome of the viral disease. The rules database comprises a number of associated rules for each available rug for treatment of the viral disease. Each rule indicates the suitability of the drug for treatment of a specific viral genotype. The patient data is entered into the rules database, the database providing an output of drugs suitable for therapy. The drugs suitable for therapy are displayed in a ranking in accordance with their suitability indication, for selection.

### SUMMARY OF THE INVENTION

In view of the foregoing, an object of the invention is to provide systems, methods and computer program products for selecting treatment regimens for patients in which available treatments are listed, and optionally ranked, based on the information of the methylation statuses at selected sites of the DNA of the patient.

A further object of the invention is to provide systems, methods and computer program products for preventive treatment regimens based on the methylation statuses at selected sites of the DNA of a patient in order to avoid or delay the acute outbreak of a disease; including metastasis of cell proliferative disorders.

As a further object of the invention, unavailable or rejected treatment regimens (e.g., regimens that would not be effective, or would be dangerous) are not displayed o r are assigned a low rank and are indicated to a user as not likely to be efficacious, or not preferred due to patient-specific complicating factors such as drug interaction from concomitant medications.

A further object of the invention is to provide systems, methods and computer program products for selecting treatment regimens and/or preventive treatment regimens based on the methylation statuses at selected sites of the DNA of a patient in which the available treatment options can be readily understood.

A further object of the invention is to provide systems, methods and computer program products for selecting treatment regimens or preventive treatment regimens based on the methylation statuses at selected sites of the DNA of a patient in which the implications of selecting a particular treatment regimen can be readily understood.

A further object of the invention is to provide systems, methods and computer program products for selecting treatment regimens based on the methylation statuses at selected sites of the DNA of a patient in which the reasons for rejection of a particular regimen can be readily understood.

A still further object of the invention is to provide systems, methods and computer program products for obtaining information about the efficacy of previous treatment regimens prescribed and/or attributed to patients.

A method of the present invention includes providing information about the methylation status at selected sites of the DNA of the patient to a computing device that includes various knowledge bases. For example, a first knowledge base may include information about a plurality of different methylation statuses at selected sites of the DNA in cells with a known disease or medical condition and/or healthy cells. It is particularly preferred that said diseases and medical conditions include subclassifications thereof, including molecular subclasses, prognostic subclasses and treatment response subclasses. In a particularly preferred embodiment this shall mean responders and non-responders to a specific treatment. It is particularly preferred that said diseases include cell proliferative disorders including cancers, neoplasms, tumors and subclassifications thereof.

A second knowledge base may include a plurality of expert rules for evaluating and selecting a type of disease or medical condition based on the methylation status at selected sites of the DNA of a patient.

A listing (preferably a ranked listing) is generated in the computing device based on the information about the methylation status at selected sites of the DNA of the patient, the first knowledge base and the second knowledge base. Based on this listing, a therapeutic treatment regimen can be recommended for the patient.

According to one embodiment of the invention, such treatment could be a preventive treatment in order to prevent the acute outbreak of a disease, in particular metastasis of cell proliferative disorders such as cancer.

In addition, in a preferred embodiment, the method further comprises a third knowledge base comprising a plurality of different therapeutic regimens for diseased cells or medical conditions a fourth knowledge base comprising a plurality of expert rules for evaluating and selecting therapeutic treatment regimens for diseased cells or medical conditions, and the step of generating in the computing device a ranked listing of available therapeutic treatment regimens for the patient based on the information generated on the basis of the third knowledge base and fourth knowledge base.

In a preferred embodiment, the method described above further includes a fifth knowledge base comprising advisory information useful for the treatment of a patient with different constituents of the different therapeutic treatment regimens and in the computing device advisory information for one or more treatment regimens in the ranked listing based on the information generated according to the method described above and the fifth knowledge base is generated.

In another embodiment of the method according to the invention, the method described above further includes the steps of entering a user-defined therapeutic treatment regimen for the disease or medical condition that is not included in the third knowledge base mentioned above and in the computing device advisory information for one or more user-defined combination therapeutic treatment regimen is generated. Preferably, the patient information in addition to the information about the m ethylation status at selected sites of the DNA may comprise gender, age, weight, hemoglobin information, neuropathy information, neutrophil information, pancreatitis, hepatic function, renal function, drug allergy and intolerance information. The patient information may further include prior therapeutic treatment regimen information. The patient information may include prior patient information stored in a computing device.

In another preferred embodiment of the inventive method, the advisory information may includes warnings to take the patient off a contraindicated drug before initiating a corresponding therapeutic treatment regimen; and information clinically useful to implement a corresponding therapeutic treatment regimen.

The method according to the present invention may comprise in the computing device a sixth knowledge base comprising patient therapeutic treatment regimen history, the advisory information including previous therapeutic treatment regimen information extracted from the sixth knowledge base.

The disease or medical condition treated by the inventive method may be a cardiovascular disease, a pulmonary disease, a neurologic disease, diabetes, a urinary tract infection, hepatitis, HIV infection, cancer or other cell proliferative disorder.

Further, in another embodiment of the inventive method drug dosage information is recommended and adjusted if necessary depending upon the patient information. Yet another method according to the invention further comprises the step of accessing, via a computing device, information for one or more therapeutic treatment regimens from a drug reference source.

The invention further provides a method for treatment of a patient with a disease or medical condition including the steps of (A) isolating a DNA-containing sample from the patient; (B) analyzing cytosine methylation patterns at selected sites of the DNA contained in the sample; (C) providing data about the methylation status at selected sites of the DNA of the patient thereby creating a first knowledge base comprising the data, a second knowledge base comprising information about a plurality of different methylation statuses at selected sites of the DNA in cells with a known disease or medical condition and/or healthy cells. It is particulary preferred that said diseases and medical conditions include subclassifications thereof, including molecular subclasses, prognostic subclasses and treatment response subclasses. In a particularly preferred embodiment this shall mean responders and non-responders to a specific treatment. It is particularly preferred that said diseases include cell proliferative disorders including cancers, neoplasms, tumors and subclassifications thereof. Preferably, the invention further comprises a third knowledge base comprising a plurality of expert rules for evaluating and selecting a type of disease or medical condition based on the methylation status at selected sites of the DNA of a patient, and the step of (D) generating a ranked listing of diseases or medical conditions based on the data of the first knowledge base, the second knowledge base and the third knowledge base. In a preferred embodiment of the inventive method, the data is provided to a computing device.

Preferably, the inventive method may include a fourth knowledge base comprising a plurality of different therapeutic regimens for diseased cells or medical conditions and a fifth knowledge base comprising a plurality of expert rules for evaluating and selecting therapeutic treatment regimens for diseased cells or medical conditions. In this case the inventive method includes the step of (E) generating a ranked listing of available therapeutic treatment regimens for the patient based on the information generated in step (D) described above and the fourth knowledge base and the fifth knowledge base.

Another method according to the invention may further include a sixth knowledge base comprising advisory information useful for the treatment of a patient with different constituents of the different therapeutic treatment regimens; and the step of (F) generating advisory information for one or more specific treatment regimens in the ranked listing based on the information generated in step (E) described above and the sixth knowledge base; and the step of (G) providing the one or more specific treatment regimens to the patient with a disease or medical condition based on the advisory information generated in step (F).

Further, in another embodiment of the inventive method, the method may further include the steps of (H) entering a user-defined therapeutic treatment regimen for the disease or medical condition that is not included in the fourth knowledge base; and (1) generating advisory information for one or more user-defined combination therapeutic treatment regimen.

The above-mentioned patient data in addition to the data about the methylation status at selected sites of the DNA comprises gender, age, weight, hemoglobin information, neuropathy information, neutrophil information, pancreatitis, hepatic function, renal function, drug allergy and intolerance information. The patient data may include prior therapeutic treatment regimen information and may include prior patient information stored in the computing device.

In yet a p referred method according to the invention the advisory information may include warnings to take the patient off a contraindicated drug before initiating a corresponding therapeutic treatment regimen; and information clinically useful to implement a corresponding therapeutic treatment regimen.

Another preferred embodiment of the method according to the invention may include a seventh knowledge base comprising patient therapeutic treatment regimen history, the advisory information including previous therapeutic treatment regimen information extracted from the seventh knowledge base. This disease or medical condition may be a cardiovascular disease, a pulmonary disease, a neurologic disease, diabetes, a urinary tract infection, hepatitis, HIV infection, cancer or other cell proliferative disorder.

In another embodiment of the method according to the invention, the drug dosage information is recommended and adjusted if necessary depending upon the patient information.

The invention further provides a method which may further include the step of accessing, via a computing device, information for one or more therapeutic treatment regimens from a drug reference source.

The invention further provides a system for guiding the selection of a therapeutic treatment regimen or a preventive therapeutic treatment regimen for a patient with a disease or medical condition. This system includes a computing device which may includes a first knowledge base comprising information about a plurality of different methylation statuses at selected sites of the DNA in cells with a known disease or medical condition and/or healthy cells. It is particularly preferred that said diseases and medical conditions include subclassifications thereof, including molecular subclasses, prognostic subclasses and treatment response subclasses. In a particularly preferred embodiment this shall mean responders and non-responders to a specific treatment. It is particularly preferred that said diseases include cell proliferative disorders including cancers, neoplasms, tumors and subclassifications thereof. The invention further comprises a second knowledge base comprising a plurality of expert rules for evaluating and selecting a type of disease or medical condition based on the methylation status at selected sites of the DNA of a patient, and preferably a third knowledge base comprising a plurality of different therapeutic regimens for diseased cells or medical conditions and a fourth knowledge base comprising a plurality of expert rules for evaluating and selecting therapeutic treatment regimens for diseased cells or medical conditions.

The system may include means for providing information about the methylation status at selected sites of the DNA of the patient to a computing device; means for generating in the computing device a ranked listing of diseases or medical conditions based on the information about the methylation status at selected sites of the DNA of the patient, the first knowledge base and the second knowledge base.

In a preferred embodiment the system according to the invention further includes a fifth knowledge base comprising advisory information useful for the treatment of a patient with different constituents of the different therapeutic treatment regimens; means for generating in the computing device a ranked listing of available therapeutic treatment regimens for the patient; and means for generating in the computing device advisory information for one or more treatment regimens in the ranked listing.

Another embodiment of the system according to the invention may further include means for entering a user-defined therapeutic treatment regimen for the disease or medical condition that is not included in the third knowledge base; and means for generating in the computing device advisory information for one or more user-defined combination therapeutic treatment regimen.

In another system according to the invention, the patient information in addition to the information about the methylation status at selected sites of the DNA may include gender, age, weight, hemoglobin information, neuropathy information, neutrophil information, pancreatitis, hepatic function, renal function, drug allergy and intolerance information. The patient information may further include prior therapeutic treatment regimen information and the patient information may include prior patient information stored in the computing device.

In another system according to the invention, the advisory information may include warnings to take the patient off a contraindicated drug before initiating a corresponding therapeutic treatment regimen; and information clinically useful to implement a corresponding therapeutic treatment regimen.

Preferably, the system according to the invention may include a computing device including a sixth knowledge base comprising patient therapeutic treatment regimen history, the advisory information including previous therapeutic treatment regimen information extracted from the sixth knowledge base.

In another system according to the invention the disease or medical condition may be a cardiovascular disease, a pulmonary disease, a neurologic disease, diabetes, a urinary tract infection, hepatitis, HIV infection, cancer or other cell proliferative disorder.

In another preferred embodiment according to the invention drug dosage information is recommended and adjusted if necessary depending upon the patient information.

Another system according to the invention may further include means for accessing, via the computing device, information for one or more therapeutic treatment regimens from a standard drug reference source.

The invention further provides a computer program product for guiding the selection of a therapeutic treatment regimen and/or a preventive therapeutic treatment regimen for a patient with a disease or medical condition. This computer program product includes a computer usable storage medium having computer readable program code means embodied in the medium, the computer readable program code means including computer readable program code means for generating a first knowledge base including information about a plurality of different methylation statuses at selected sites of the DNA in cells with a known disease or medical condition and/or healthy cells. It is particularly preferred that said diseases and medical conditions include subclassifications thereof, including molecular subclasses, prognostic subclasses and treatment response subclasses. In a particularly preferred embodiment this shall mean responders and non-responders to a specific treatment. It is particularly preferred that said diseases include cell proliferative disorders including cancers, neoplasms, tumors and subclassifications thereof. Computer readable program code means further comprise a second knowledge base including a plurality of expert rules for evaluating and selecting a type of disease or medical condition based on the methylation status at selected sites of the DNA of a patient, and preferably a third knowledge base comprising a plurality of different therapeutic regimens for diseased cells or medical conditions, a fourth knowledge base including a plurality of expert rules for evaluating and selecting therapeutic treatment regimens for diseased cells or medical conditions, a fifth knowledge base comprising advisory information useful for the treatment of a patient with different constituents of the different therapeutic treatment regimens; and computer readable program code means for providing information about the methylation status at selected sites of the DNA of the patient; computer readable program code means for generating a ranked listing of diseases or medical conditions based on the information about the methylation status at selected sites of the DNA of the patient; and computer readable program code means for generating in the computing device a ranked listing of available therapeutic treatment regimens for the patient.

A preferred embodiment of the computer program product according to the invention may further include computer readable program code means for generating in the computing device advisory information for one or more treatment regimens in the ranked listing. The computer program product according to the invention may further include computer readable program code means entering a user-defined therapeutic treatment regimen for the disease or medical condition that is not included in the third knowledge base; and computer readable program code means for generating in the computing device advisory information for one or more user-defined combination therapeutic treatment regimen.

Preferably, the computer program product according to the invention, the patient information in addition to the information about the methylation status at selected sites of the DNA may include gender, age, weight, hemoglobin information, neuropathy information, neutrophil information, pancreatitis, hepatic function, renal function, drug allergy and intolerance information. The patient information may include prior therapeutic treatment regimen information and the patient information may further include prior patient information.

In another preferred embodiment of the computer program product according to the invention, the advisory information may include warnings to take the patient off a contraindicated drug before initiating a corresponding therapeutic treatment regimen; and information clinically useful to implement a corresponding therapeutic treatment regimen.

In another embodiment of the computer program product according to the invention the computer readable program code means may include computer readable program code means for generating a sixth knowledge base comprising patient therapeutic treatment regimen history, the advisory information including previous therapeutic treatment regimen information extracted from the sixth knowledge base.

In another preferred embodiment of the computer program product according to the invention, the disease or medical condition may be a cardiovascular disease, a pulmonary disease, a neurologic disease, diabetes, a urinary tract infection, hepatitis, HIV infection, cancer or other cell proliferative disorder.

Further, in another preferred embodiment of a computer program product according to the invention, drug dosage information is recommended and adjusted if necessary depending upon the patient information.

The inventive computer program product according to the invention may further include computer readable program code means for accessing information for one or more therapeutic treatment regimens from a standard drug reference source.

Further objects and aspects of the present invention are explained in detail in the drawings herein and the specification set forth below.

### BRIEF DESCRIPTION OF THE DRAWINGS

The accompanying drawings, which are incorporated in and constitute a part of the specification, illustrate embodiments of the invention and, together with the description, serve to explain principles of the invention.

FIG. 1 illustrates a process of the present invention, including routines for entering data with respect to the methylation status at specific sites of the patients' DNA, therapeutic treatment regimen and preventive therapeutic treatment regimen.

FIG. 2 schematically illustrates a system or apparatus of the present invention.

FIG. 3 illustrates a client-server environment within which the system of FIG. 2 may operate, according to an embodiment of the present invention, and wherein a central server is accessible by at least one local server via a computer network, such as the Internet, and wherein each local server is accessible by at least one client.

### DETAILED DESCRIPTION OF THE INVENTION

The present invention now will be described more fully hereinafter with reference to the accompanying drawings, in which preferred embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art. Like numbers refer to like elements throughout.

As will be appreciated by one of skill in the art, the present invention may be embodied as a method, data processing system, or computer program product. Accordingly, the present invention may take the form of an entirely hardware embodiment, an entirely software embodiment, or an embodiment combining software and hardware aspects. Furthermore, the present invention may take the form of a computer program product on a computer-usable storage medium having computer readable program code means embodied in the medium. Any suitable computer readable medium may be utilized including, but not limited to, hard disks, CD-ROMs, optical storage devices, and magnetic storage devices.

The present invention is described below with reference to flowchart illustrations of methods, apparatus (systems), and computer program products according to an embodiment of the invention. It will be understood that each block of the flowchart illustrations, and combinations of blocks in the flowchart illustrations, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions specified in the flowchart block or blocks.

These computer program instructions may also be stored in a computer-readable memory that can direct a computer or other programmable data processing apparatus to function in a particular manner, such that the instructions stored in the computer-readable memory produce an article of manufacture including instruction means which implement the function specified in the flowchart block or blocks.

The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions specified in the flowchart block or blocks.

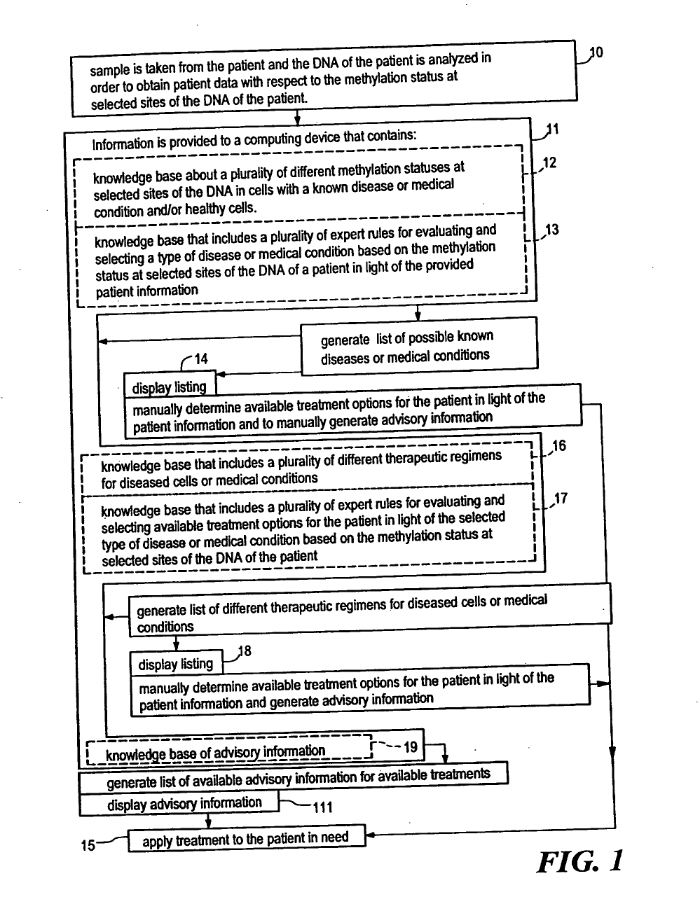

A method of the instant invention is illustrated in FIG. 1. In the first step 10, a sample to be analyzed is taken from the patient and the DNA of the patient is analyzed in order to obtain patient data with respect to the methylation status at selected sites of the DNA of the patient. This information is then provided to a computing device 11. The patient may be further examined to obtain further patient information that may include one or more of gender, age, weight, CD4.sup.+cell information, viral load information, HIV genotype and phenotype information, hemoglobin information, neuropathy information, neutrophil information, pancreatitis, hepatic function, renal function, drug allergy and intolerance information, and information for drug treatments for other conditions. The information may include historical information on prior therapeutic treatment regimens for the disease or medical condition. While the patient is typically examined on a first visit to determine the patient information, it will be appreciated that patient information may also be stored in the computing device, or transferred to the computing device from another computing device, storage device, or hard copy, when the information has been previously determined.

The patient information is usually provided to a computing device 11 that contains a knowledge base about a plurality of different methylation statuses at selected sites of the DNA in cells with a known disease or medical condition and/or healthy cells 12 and a knowledge base that includes a plurality of expert rules for evaluating and selecting a type of disease or medical condition based on the methylation status at selected sites of the DNA of a patient in light of the provided patient information 13.

A list (preferably a ranked list) is then generated in the computing device based on the information about the methylation status at selected sites of the DNA of the patient, the knowledge base about the different methylation statuses at selected sites of the DNA in cells with a known disease or medical condition and/or healthy cells and the plurality of expert rules for evaluating and selecting a type of disease or medical condition based on the methylation status at selected sites of the DNA of a patient.

This ranked list indicates all possible known diseases or medical conditions of the patient and can be displayed 14. In one embodiment of the invention, the displayed information is then used to manually determine available treatment options for the patient in light of the patient information and to manually generate advisory information. Based on the information displayed at 14, a treatment can be applied to the patient in need 15.

The method illustrated in FIG. 1 further includes a knowledge base 16 that includes a plurality of different therapeutic regimens for diseased cells or medical conditions and a knowledge base 17 that includes a plurality of expert rules for evaluating and selecting available treatment options for the patient in light of the selected type of disease or medical condition based on the methylation status at selected sites of the DNA of the patient.

A list (preferably a ranked list) is then generated in the computing device based on the knowledge base 16 that includes a plurality of different therapeutic regimens for diseased cells or medical conditions and a knowledge base 17 that includes a plurality of expert rules for evaluating and selecting available treatment options for the patient in light of the selected type of disease or medical condition based on the methylation status at selected sites of the DNA of the patient.

This ranked list indicates all possible known therapeutic regimens for diseased cells or medical conditions for the patient and can be displayed 18. In another embodiment of the invention, the displayed information is then used to manually determine available treatment options for the patient in light of the patient information and to manually generate advisory information. Based on the information displayed at 18, a treatment can be applied to the patient in need 15.

The method also includes a knowledge base of advisory information 19. A list of available advisory information for the available treatments is then generated on the basis of the knowledge base of advisory information and the list that indicates all possible known therapeutic regimens and displayed 111. The advisory information may include warnings to take the patient off a contraindicated drug or select a suitable non contraindicated drug to treat the condition before initiating a corresponding treatment regimen and/or information clinically useful to implement a corresponding therapeutic treatment regimen. Based on the information displayed either at 14, 18 or 111, a treatment is applied to the patient in need 15. The progress of this treatment can be constantly monitored by taking intermediate DNA samples from the patient and performing an analysis of the changes of the methylation statuses of the patient similar to the method described above.

In another embodiment of the invention, the information displayed either at 14, 18 or 111 is used to apply a preventive treatment to the patient in order to prevent the acute out-break of a disease in particular metastasis of cancer or other cell proliferative disorders.

Diseases (or medical conditions), the treatment of which may be facilitated or improved by the present invention, are those for which multiple different therapy options are available for selection and treatment. Such diseases and medical conditions may include, but are not limited to, cardiovascular disease (including but not limited to congestive heart failure, hypertension, hyperlipidemia and angina), pulmonary disease (including but not limited to chronic obstructive pulmonary disease, asthma, pneumonia, cystic fibrosis, and tuberculosis), neurologic disease (including but not limited to Alzheimer's disease, Parkinson's disease, epilepsy, multiple sclerosis, amyotrophic lateral sclerosis or ALS, psychoses such as schizophrenia and organic brain syndrome, neuroses, including anxiety, depression and bipolar disorder), hepatitis infections (including hepatitis B and hepatitis C infection), urinary tract infections, venereal disease, cancer (including but not limited to breast, lung, prostate, and colon cancer), etc. It should be appreciated that prevention of development or onset of the above-mentioned diseases and medical conditions may be facilitated or improved by the present invention.

The present invention is also useful for cases in which the disease of the patient is known, for example such as cancer, or where the known disease is any medical condition for which a combination therapeutic treatment regimen can be used. The invention is particularly useful when the list of available treatments includes a plurality (e.g., 2, 10 or 15 or more) of treatment, combination therapeutic treatment regimens (e.g., therapeutic treatment regimens incorporating two or more active therapeutic agents), where the potential for drug interactions is increased and/or the complexity involved in selecting the best available treatment is multi-factional. The invention is also particularly useful in the treatment of cancers and such diseases wherein only a small subset of patients may benefit from a particular therapy, where a large number of therapeutic options are available and wherein the side-effects of treatment may be highly undesirable or life threatening.

Alternatively, the advisory information can be generated automatically for non-recommended therapeutic treatment regimens. These various steps can be repeated in any sequence in an interactive manner to provide the user with assurance that all treatment options have been given adequate and appropriate consideration.

The terms “therapy” and “therapeutic treatment regimen” are interchangeable herein and, as used herein, mean any pharmaceutical or drug therapy, regardless of the route of delivery (e.g., oral, intraveneous, intramuscular, subcutaneous, intraarterial, intraperitoneal, intrathecal, etc.), for any disease (including both chronic and acute medical conditions, disorders, and the like). In addition, it is understood that the present invention is not limited to facilitating or improving the treatment of diseases. The present invention may be utilized to facilitate or improve the treatment of patients having various medical conditions, without limitation. The term also includes preventive therapeutic treatment regimens, which may be applied in order to prevent an outbreak of an acute disease.

The term ‘metastasis’ as used herein shall be taken to mean the transfer of a disease-producing agent (such as bacteria, cancer or other cell proliferative disorder cells) from an original site of disease to another part of the body with development of a similar lesion in the new location.

The term ‘medical condition’ as used herein shall be taken to mean injuries, disabilities, syndromes, symptoms, deviant behaviors, and atypical physiological characteristics including variations of structure, response and function. In particular the term shall be taken to include differential therapeutic treatment response of diseased patients including those caused by genetic and/or epigenetic predisposition, for example but not limited to response (an improvement related to treatment), non-response and adverse reaction to a therapeutic treatment.

The term ‘disease’ as used herein shall be taken to include all subclassifications thereof, for example but not limited to type, location, stage, molecular, epigenetic, genetic, prognostic, pathological, immunological, cytologic subclasses of a disease. Furthermore said term shall include physiological variants of a disease including differential therapeutic treatment response of diseased patients including those caused by genetic and/or epigenetic pre-disposition, for example but not limited to response (an improvement related to treatment), non-response and adverse reaction to a therapeutic treatment.

The term ‘prognostic subclasses’ as used herein shall be taken to mean subclasses of a disease with differential likely course and outcome which may be measured according to standards commonly used in the art, including progression-free survival, recurrence-free survival, disease free survival, life expectancy and mortality rates.

A “knowledge base” according to the present invention consists of criteria for the evaluation and selection of treatment regimens for the diagnosed disease condition from the third knowledge base. Each therapy or combination of therapies is evaluated according to one or more medication selection criteria, including, but not limited to: a) Efficacy of the treatment; b) Cost; c) Adverse drug reactions; d) Drug interactions including drug-drug, drug-food and drug-disease; e) Allergic reactions; f) Other contraindications; g) Preferred drugs contained in a drug benefits plan issued by a drugs benefit provider to a given patient, and/or h) Patient history (e.g. Body system function tests, for example renal or liver function tests). All these criteria belong to the standard set of criteria that is well known to the person of skill, e.g. the attending physician. The selection of medication selection criteria and the relative importance of each medication selection criteria in the evaluation being defined by the clinician or other person. The clinician or other person may further define medication selection criteria for the exclusion of treatment regimens from the evaluation (for example, all treatments above a certain cost, all treatments to which the patient has a known allergy and/or all treatments which affect liver function in a patient with poor liver function).

It is further preferred that each medication selection criteria may be given a relative weighting as compared to the other medication selection criteria e.g. if one of the medication selection criteria is considered to be particularly important it would be given a higher weighting than a less important medication selection criteria. Each therapy or combination of therapies is then evaluated according to each of the selected medication selection criteria wherein each medication selection criteria is given a score. The therapy or combination of therapies may then be ranked by the sum score of one or more medication selection criteria.

In another embodiment, it could increase the quality and flexibility of the overall decision to introduce a ranked listing, in which not only the best quality disease is displayed, as the selection is made from a list that is constantly built up, that is, in which additional and yet unknown diseases and their complex methylation statuses are added. The list is made from the analyses as introduced in the first knowledge base, as the base is compared with the result of the sample to be analysed. The list could contain information as provided in the publications as mentioned herein above in the present specification.

The statistical methods employed for the present invention may be either multivariate statistical methods or univariate statistical methods. The suitability of each method will be apparent to one skilled in the art. For example, in one embodiment of the method according to the invention, said method is characterized in that for each patient the statistical distance of the methylation status at selected sites of the DNA (hereinafter also referred to as the “methylation profile” or “methylation pattern” or “methylation status”) from the methylation profiles of known diseases, medical conditions and/or healthy states are calculated and wherein a deviation is beyond a pre-determined limit said disease, medical condition or healthy state is excluded from the ranking.

“Selecting a type of disease or medical condition based on the methylation status” involves first the analysis of the methylation pattern of the DNA of a sample derived from subject suspected to have a certain disease, i.e. the subject under analysis. Said sample can be derived from any suitable source, e.g. blood, urine, stool, biopsies, histological slides, etc. as long as it contains DNA to be analysed. The analysis can be performed using methods as described herein. During said analysis the methylation status of CpG dinucleotides can be determined in a “shotgun” fashion, i.e. random methylation sites are analysed. This analysis can be performed using, e.g. a chip technology, wherein the CpGs of the genes as present on the chip will, optimally, represent a statistical distribution of CpGs in the genes that are usually expressed in the chromosome. Alternatively, the analysis can be performed at certain specific sites of the genome, wherein the analysis is performed based on an initial physical examination of the patient. As an example, a subject exhibiting the general symptoms of leukemia (as well known in the art) will be subjected to an analysis at sites known to be differently methylated in healthy and diseased patients. In addition, the same analysis as with the sample of the person under analysis will be performed with a sample of a healthy subject (or the data of the methylation analysis of a healthy person is used for the analysis).

The datasets of the methylation patterns are then compared. Based on this comparison, the differences in the pattern(s) between the healthy subject and the person under analysis will be used to generate a “disease specific methylation pattern” of the subject under analysis.

The term “statistical distance” is taken to mean a distance between the single measurement vector (the methylation pattern to be classified) and the variety of methylation patterns stored in the database that each belong to a disease, medical condition (of diseases as described above) or healthy state, (referred to as “reference data set” in the following). The statistical distance is calculated with respect to the statistical distribution of the reference data set. It is particularly preferred that the method according to the invention is implemented by means of a computer. The disease, medical condition or healthy state are then ranked according to their statistical distance from the methylation profile of the patient, most preferably in ascending order (i.e. the disease, medical condition or healthy state which has the closest statistical distance to the methylation profile of the patient is the most highly ranked).

The statistical distance may be calculated by means of one or more methods taken from the group consisting or the Hotelling's T2 distance or cross-correlation between a single test measurement vector and the reference data set. Furthermore, a statistical distance can be expressed as a score that is the linear or nonlinearly transformed weighted sum of methylation status of certain selected CpGs, where the weights and the non-linear transformation have been determined previously, using the reference data sets as training samples by any supervised learning method known for those who is skilled in the art.

Based on the statistical differences of the diseases, based on the information of the methylation status of the patient under analysis, a ranked listing of diseases is generated, which reflects the likelihood with respect to the disease or medical condition the patient is suffering from. Said listing is therefore generated from a combination of the specific methylation pattern of the patient under analysis and the knowledge base of disease specific methylation patterns that has been generated based on the methylation patterns of earlier diagnosed diseases.

System Description

The present invention may be embodied as an expert system that provides decision support to physicians (or other health care providers) treating patients with a known or unknown disease, such as an infection. A system according to the present invention analyses the methylation statuses at selected sites of the DNA of a patient, attaches this information to other knowledge bases and calculates therapy options and/or preventive therapy options and attaches all relevant information to those options.

As known to those of skill in the art, an expert system, also known as artificial intelligence (AI), is a computer program that can simulate the judgement and behavior of a human or an organization that has expert knowledge and experience in a particular field. An expert system typically contains a knowledge base containing accumulated experience and a set of rules for applying the knowledge base to each particular situation that is described to the program. Another expert system is known as neuronal network (NN) which is capable to actively accumulate information and knowledge. Other expert systems are well known to those of skill in the art and need not be described further herein.

As an example, the antibacterial and/or anti-retroviral therapy options (combinations of antiretroviral drugs), are derived using a knowledge base consisting of a number of expert system rules and functions which in turn take into account a given patient's treatment history, current condition and laboratory values. A system according to the present invention supports the entry, storage, and analysis of patient data with respect to the methylation statuses at selected sites of the DNA of the patient in a large central database. A system according to the present invention has a flexible data driven architecture and custom reporting capabilities designed to support patient therapy management and clinical drug trial activities such as screening, patient tracking and support. It is anticipated that a system according to the present invention may be used by health care providers (including physicians), clinical research scientists, and possibly healthcare organizations seeking to find the most cost-effective treatment options for patients in general as well as the most effective treatment regimen and/or preventive treatment regimen for the individual patient while providing the highest standard of care.

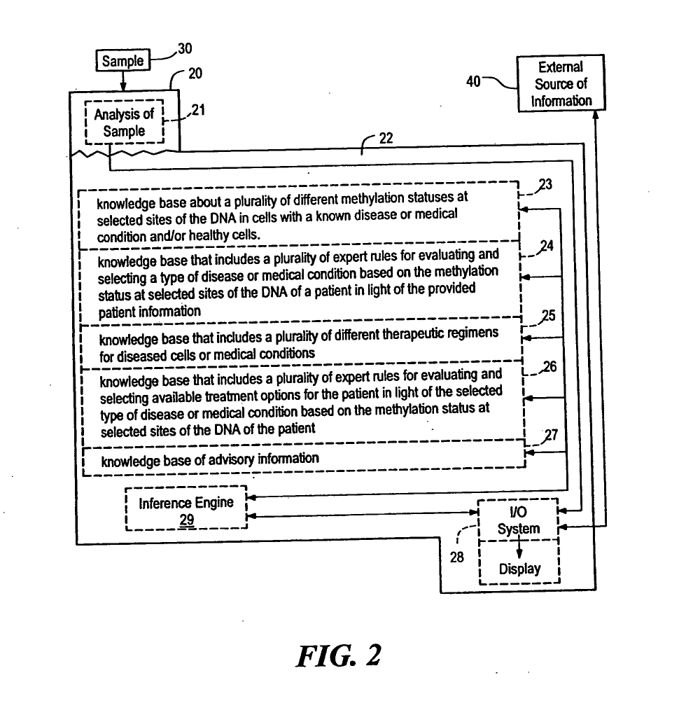

A system 20 for carrying out the present invention is schematically illustrated in FIG. 2. The system 20 consists out of two major components 21 and 22. The first component 21 is capable of analyzing a sample 30 of the patient for its methylation statuses of the DNA at selected sites. Such component comprises, for example, apparatuses for PCR, mass spectrometry, and/or electrophoresis, roboters which automatically handle the sample to be analyzed during the analysis procedure together with components which are capable of converting the generated information into computer readable signals. The second component 22 is capable of generating and displaying information about the type of disease of the patient and/or advisory information with respect to an individual (preventive) treatment regimen for the patient.

The second component 22 comprises a first knowledge base 23 comprising information about a plurality of different methylation statuses at selected sites of the DNA in cells with a known disease or medical condition and/or healthy cells. It is particularly preferred that said diseases and medical conditions include subclassifications thereof, including molecular subclasses, prognostic subclasses and treatment response subclasses. In a particularly preferred embodiment this shall mean responders and non-responders to a specific treatment. It is particularly preferred that said diseases include cell proliferative disorders including cancers, neoplasms, tumors and subclassifications thereof. The second component 22 further comprises a second knowledge base 24 comprising a plurality of expert rules for evaluating and selecting a type of disease or medical condition based on the methylation status at selected sites of the DNA of a patient, a third knowledge base 25 comprising a plurality of different therapeutic regimens and/or preventive therapeutic treatment regimens for diseased cells or medical conditions, which may be ranked for efficacy (e.g., by a panel of experts) or ranked according to system rules, a fourth knowledge base 26 comprising a plurality of expert rules for evaluating and selecting therapeutic treatment regimens for diseased cells or medical conditions and a fifth knowledge base 27 comprising advisory information useful for the treatment of a patient with different constituents of said different therapeutic treatment regimens. Optionally, the second component comprises a knowledge base (not shown) of patient therapy history and additional patient information.

The “expert rules” according to the present invention consist of “statistical methods” and/or computer program products that are suitable for the comparison of the methylation status at selected sites of the DNA of the patient to a knowledge base comprising information about a plurality of different methylation statuses at selected sites of the DNA in cells with a known disease or medical condition and/or healthy cells (herein also referred to the “methylation profile” of the known disease, medical condition or healthy state).

The statistical methods may be either multivariate statistical methods or univariate statistical methods. The suitability of each method will be apparent to one skilled in the art. For example, in one embodiment of the method according to the invention, said method is characterized in that for each patient the statistical distance of the methylation status at selected sites of the DNA (hereinafter also referred to as the “methylation profile”) from the methylation profiles of known diseases, medical conditions and/or healthy states are calculated and wherein a deviation is beyond a pre-determined limit said disease, medical condition or healthy state is excluded from the ranking.

The term “statistical distance” is taken to mean a distance between the single measurement vector (the methylation pattern to be classified) and the variety of methylation patterns stored in the database that each belong to a disease, medical condition or healthy state, (referred herein to as “reference data set”). The statistical distance is calculated with respect to the statistical distribution of the reference data set. It is particularly preferred that the method according to the invention is implemented by means of a computer. The disease, medical condition or healthy state are then ranked according to their statistical distance from the methylation profile of the patient, most preferably in ascending order (i.e. the disease, medical condition or healthy state which has the closest statistical distance to the methylation profile of the patient is the most highly ranked).

The statistical distance may be calculated by means of one or more methods taken from the group consisting or the Hotelling's T2 distance or cross-correlation between a single test measurement vector and the reference data set. Furthermore, a statistical distance can be expressed as a score that is the linear or non-linearly transformed weighted sum of methylation status of certain selected CpGs, where the weights and the non-linear transformation have been determined previously, using the reference data sets as training samples by any supervised learning method known for those who is skilled in the art.

A “knowledge base” according to the present invention consists of criteria for the evaluation and selection of treatment regimens for the diagnosed disease condition from the third knowledge base. Each therapy or combination of therapies is evaluated according to one or more medication selection criteria, including, but not limited to: a) Efficacy of the treatment; b) Cost; c) Adverse drug reactions; d) Drug interactions including drug-drug, drug-food and drug-disease; e) Allergic reactions; f) Other contraindications; g) Preferred drugs contained in a drug benefits plan issued by a drugs benefit provider to a given patient, and/or h) Patient history (e.g. Body system function tests, for example renal or liver function tests). All these criteria belong to the standard set of criteria that is well known to the person of skill, e.g. the attending physician. The selection of medication selection criteria and the relative importance of each medication selection criteria in the evaluation being defined by the clinician or other person. The clinician or other person may further define medication selection criteria for the exclusion of treatment regimens from the evaluation (for example, all treatments above a certain cost, all treatments to which the patient has a known allergy and/or all treatments which affect liver function in a patient with poor liver function).

It is further preferred that each medication selection criteria may be given a relative weighting as compared to the other medication selection criteria e.g. if one of the medication selection criteria is considered to be particularly important it would be given a higher weighting than a less important medication selection criteria. Each therapy or combination of therapies is then evaluated according to each of the selected medication selection criteria wherein each medication selection criteria is given a score. The therapy or combination of therapies may then be ranked by the sum score of one or more medication selection criteria.

In another embodiment, it could increase the quality and flexibility of the overall decision to introduce a ranked listing, in which not only the best quality disease is displayed, as the selection is made from a list that is constantly built up, that is, in which additional and yet unknown diseases and their complex methylation statuses are added. The list is made from the analyses as introduced in the first knowledge base, as the this base is compared with the result of the sample to be analysed. The list could contain information as provided in the publications as mentioned herein above in the present specification, nevertheless, these have to seen in a complex fashion of analysis.

Other ways of determining “expert rules” are also known in the state of the art and said expert rules could be easily modified by the skilled artisan in order to be applied in accordance with the present invention. Examples are U.S. Pat. No. 6,188,988 and U.S. Pat. No. 6,081,786 (“Systems, methods and computer program products for guiding the selection of therapeutic treatment regimens”), U.S. Pat. No. 5,537,590 (“Apparatus for applying analysis rules to data sets in a relational database to generate a database of diagnostic records linked to the data sets”), U.S. Pat. No. 5,511,004 (“Diagnostic method for an evolutionary process”), and U.S. Pat. No. 5,485,610 (“Physical database design system”) which are herewith specifically incorporated by reference for the purposes of the present invention.

Patient information is preferably stored within a database and is configured to be updated. The knowledge bases and patient information may be updated by an input/output system 28, which can comprise a keyboard (and/or mouse) and video monitor. Note also that, while the knowledge bases and patient data are shown as separate blocks, the knowledge bases and patient data can be combined together (e.g., the expert rules and the advisory information can be combined in a single database).

To carry out the method described above, the information from at least two of blocks 23-27 is provided to an inference engine 29, which generates the listing of either diseases of the patient or of available treatments and the corresponding advisory information from the information provided by the blocks.

The inference engine may be implemented as hardware, software, or combinations thereof. Inference engines are known and any of a variety thereof may be used to carry out the present invention. Examples include, but are not limited to, those described in U.S. Pat. No. 5,263,127 to Barabash et al. (Method for fast rule execution of expert systems); U.S. Pat. No. 5,720,009 to Kirk et al. (Method of rule execution in an expert system using equivalence classes to group database objects); U.S. Pat. No. 5,642,471 to Paillet (Production rule filter mechanism and inference engine for expert system); U.S. Pat. No. 5,664,062 to Kim (High performance max-min circuit for a fuzzy inference engine).

High-speed inference engines are preferred so that the results of data entered are continually updated as new data is entered. As with the knowledge bases and patient information in blocks, the inference engine may be a separate block from the knowledge bases and patient information blocks, or may be combined together in a common program or routine. Optionally, exterior knowledge bases can be used as well. The information that is generated in the inference engine can then be displayed via an input/output system. Based on the displayed information, the person in charge of the medical supervision of the patient will be able to select and apply a therapeutical treatment regimen to the patient. At any time, feedback information on types of diseases, success of the treatment regimen and available medicaments can be added via the input/output system. Optionally, this data can be supplied from an external source 40, e.g. a remote server.

Note that the advisory information that is generated for any available therapy may differ from instance to instance based on differences in the patient information provided.

System Architecture

The present invention can be implemented as a system which comprises a first component 21 able to perform an analysis of the methylation statuses of the DNA of the patient. This device is capable of extracting the DNA from the patients' sample provided to said component and to perform several analytical steps in order to receive the methylation statuses at selected sites of the DNA. These analytical steps are known to the person skilled in the art and may comprise bisulfite treatments, amplification cycles of the DNA employing polymerase chain reaction (PCR) protocols and reactions, hybridisation reactions, DNA sequencing, mass spectroscopy or measurements of fluorescence. All parts of the first device are usually combined and arranged in such a way that the procedure will be as much automated as possible in order to avoid human mistakes and create a high-throughput environment.

The data generated in the first component is provided to a second component 22 which is able to perform calculations using the provided data in order to generate information relevant for the following therapeutic treatment of the patient. The second component can be implemented as a system running on a stand alone computing device.

Preferably, the present invention is implemented as a system in a client-server environment. As is known to those of skilled in the art, a client application is the requesting program in a client-server relationship. A server application is a program that awaits and fulfills requests from client programs in the same or other computers. Client-server environments may include public networks, such as the Internet, and private networks often referred to as “intranets”, local area networks (LANs) and wide area networks (WANs), neural networks (NN), virtual private networks (VPNs), frame relay or direct telephone connections. It is understood that a client application or server application, including computers hosting client and server applications, or other apparatus configured to execute program code embodied within computer usable media, operates as means for performing the various functions and carries out the methods of the various operations of the present invention. In one preferred embodiment of the invention, the results of the calculation of the client can also be reported back to the server.

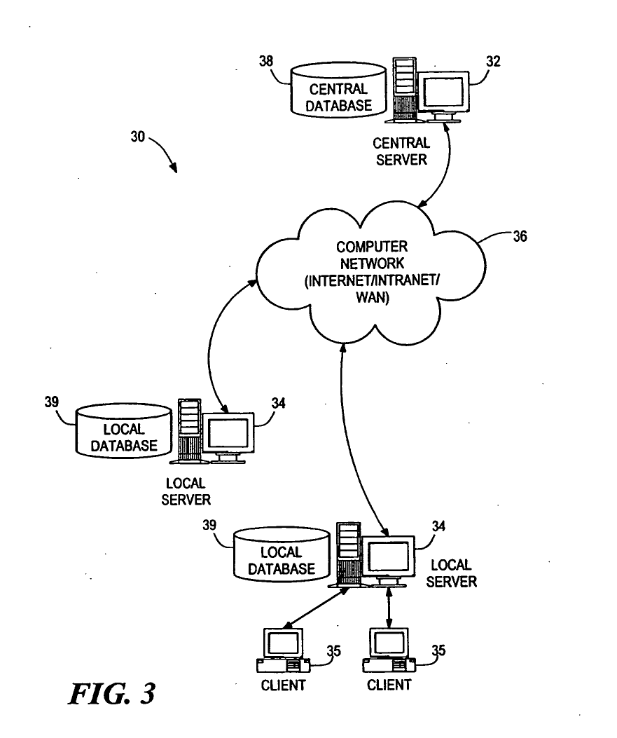

Referring now to FIG. 3, a client-server environment 30 according to a preferred embodiment of the present invention is illustrated. The illustrated client-server environment 30 includes a central server 32 that is accessible by at least one local server 34 via a computer network 36, such as the Internet. A variety of computer network transport protocols including, but not limited to TCP/IP, can be utilized for communicating between the central server 32 and the local servers 34.

Central Server

The central server 32 includes a central database 38, such as the Microsoft.RTM. SQL Server application program, version 6.5 (available from Microsoft, Inc., Redmond, Wash.), executing thereon. The central server 32 ensures that the local servers 34 are running the most recent version of a knowledge base. The central server 32 also stores all patient data and data on methylation patterns and possible treatment regimens and performs various administrative functions including adding and deleting local servers and users to the system (20, FIG. 2). The central server 32 also provides authorization before a local server 34 can be utilized by a user. Patient data and/or data of the methylation statuses at selected sites of the DNA of patients, also called “methylation patterns” herein, is preferably stored on the central server 32, thereby providing a central repository of patient data and methylation data. However, it is understood that patient data can be stored on a local server 34 or on local storage media. Data on patients and methylation patterns as well as data with respect to therapeutical treatment regimens can be submitted to the central server from the local servers or from a central device creating data on methylation patterns, e.g. at a laboratory that analyses samples of patients on a large scale and supplies the data of multiple methylation patterns of multiple patients to the central server.

Local Server

Each local server 34 typically serves multiple users in a geographical location. Each local server 34 includes a server application, an inference engine, one or more knowledge bases, and a local database 39. Each local server 34 performs artificial intelligence processing for carrying out operations of the present invention. When a user logs on to a local server 34 via a client 35, the user is preferably authenticated via an identification and password, as would be understood by those skilled in the art. Once authenticated, a user is permitted access to the system (20, FIG. 2) and certain administrative privileges are assigned to the user.

Each local server 34 also communicates with the central server 32 to verify that the most up-to-date version of the knowledge base(s) and application are running on the requesting local server 34. If not, the requesting local server 34 downloads from the central server 32 the latest validated knowledge base(s) and/or application before a user session is established. Once a user has logged onto the system (20, FIG. 2) and has established a user session, all data and artificial intelligence processing is preferably performed on a local server 34. An advantage of the illustrated client-server configuration is that most of the computationally intensive work occurs on a local server 34, thereby allowing “thin” clients 35 (i.e., computing devices having minimal hardware) and optimizing system speed.

In a preferred embodiment, each local server database 39 is implemented via a Microsoft™ SQL Server application program, Version 6.5. The primary purpose of each local database 39 is to store various patient identifiers and to ensure secure and authorized access to the system (20, FIG. 2) by a user. It is to be understood, however, that both central and local databases 38, 39 may be hosted on the central server 32.

Local Client

Each local client 35 also includes a client application program that consists of a graphical user interface (GUI) and a middle layer program that communicates with a local server 34. Program code for the client application program may execute entirely on a local client 35, or it may execute partly on a local client 35 and partly on a local server 34. As will be described below, a user interacts with the system (20, FIG. 2) by providing a sample of the patient to the first component (21, FIG. 2) of the system (20, FIG. 2) and optionally entering (or accessing) patient data within a GUI displayed within the client 35. The client 35 then communicates with a local server 34 for analysis of the patient information with respect to the methylation status of the DNA of the patient at selected sites of the patients' DNA which is generated in the system (20, FIG. 2) and/or entered via the GUI.

Computer program code for carrying out operations of the present invention is preferably written in an object oriented programming language such as JAVA.RTM., Smalltalk, or C++. However, the computer program code for carrying out operations of the present invention may also be written in conventional procedural programming languages, such as the “C” programming language, in an interpreted scripting language, such as Perl, or in a functional (or fourth generation) programming language such as Lisp, SML, or Forth.

The middle layer program of the client application includes an inference engine within a local server 34 that provides continuous on-line direction to users, and can instantly warn a user when a patient is assigned drugs or a medical condition that is contraindicated with, or antagonistic of, the patient's current therapy.

Inference Engine

Inference engines are well known by those of skill in the art and need not be described further herein. Each knowledge base used by an inference engine according to the present invention is a collection of rules and methods authored by a clinical advisory panel of disease-treating physicians and scientists. A knowledge base may have subjective rules, objective rules, and system-generated rules. Objective rules are based on industry established facts regarding the treatment of diseases using drug therapy and are drawn from the package insert information of drug manufacturers and from peer reviewed and published journal articles.

For objective rules, the present invention can be configured so as to prevent a user from receiving recommendations on new therapy options when certain crucial data on the patient has not been entered. However, it is understood that the present invention does not prevent a health care provider, such as a physician, from recording his/her therapy decisions, even if the system (20, FIG. 2) has shown reasons why that therapy may be harmful to the patient. The present invention allows a health care provider to be the final authority regarding patient therapy.

Subjective rules are based on expert opinions, observations and experience. Subjective rules are typically developed from “best practices” information based on consensus opinion of experts in the field. Such expert opinion may be based on knowledge of the literature published or presented in the field or their own experience, from clinical practice, research or clinical trials of approved and unapproved medications. A number of experts are used so that personal bias is reduced.

System generated rules are those derived from the outcomes of patients tracked in the system who received known and defined therapies and either improved, stabilized or worsened during a defined period. Because of the large number of potential combinations useable in infections, this system generated database and rules derived from them are likely to encompass data beyond that achievable from objective or subjective rules databases.

The following non-limiting examples illustrate various aspects of the present invention. These examples are provided for illustrative purposes only, and are not intended to be limiting of the invention.

### EXAMPLE 1

Example: Diagnosis and Treatment of Patient with a Colon Cell Proliferative Disorder

For the initial build-up of the database system for use in the method of the present invention, a first knowledge base is generated by collecting information regarding the methylation status of selected sites of a DNA, wherein said methylation status is correlated with certain diseases or medical conditions. Furthermore, comparative data is added that reflects the methylation status of the same selected sites in a sample taken from a healthy individual. The initial connective data between the methylation statuses and the respective diseases (and/or specific subtypes of diseases, such as different forms of colon cell proliferative disorders or other cancerous diseases) is generated by connecting a specific disease type that has been diagnosed based on physiological diagnostic factors of the patient (such as the histological analysis of tissues or the analysis of blood cells) and characterised by the methylation patterns of marker genes with the methylation status of said diagnosed patient at selected sites of the DNA of said patient.

For such an analysis, either the methylation statuses at sites can be analyzed that are potentially methylated in genes that are known or suspected to play a role in the development or progression of the specific disease, or a general analysis of disease sites in the DNA of the patient can be performed to achieve a so called “fingerprint” of the methylation status of genes in the patient (for example, using a chip that is suitable for the analysis of CpGs in genes that are usually expressed in, e.g., a liver cell). Once this first knowledge base has been generated, it will be integrated into a computing device.

In the following, example the CpG methylation levels of the following genes or their promoter regions are stored in the form of a database:

| Sequence | Genbank Ref. | |
|---|---|---|
| number | ID No:/contig | Description |
| 1 | AL355481. | Hypothetical protein-leucine rich repeat |
| | 12.1.122435 | (Vega gene ID |
| | | OTTHUMG00013001328) |
| 2 | AC027601. | 5 azacytidine induced (Ensembl gene ID |
| | 25.1.192732 | 141577) |
| 3 | AL831711. | No known gene, close to ATP/GTP- |
| | 4.1.8801 | binding site motif A (P-loop) protein |
| 4 | NM_002938 | RING FINGER PROTEIN 4; RNF4 |
| 5 | NM_021926 | HOMEOBOX PROTEIN ARISTALESS- |
| | | LIKE 4;. ALX 4 |
| 6 | NM_004852 | ONE CUT DOMAIN FAMILY MEMBER |
| | | 2; ONC2 |
| 7 | NM_002507 | TUMOR NECROSIS FACTOR |
| | | RECEPTOR SUPERFAMILY MEMBER |
| | | 16 PRECURSOR (LOW-AFFINITY |
| | | NERVE GROWTH FACTOR |
| | | RECEPTOR) (NGF RECEPTOR) (GP80- |
| | | LNGFR) (P75 ICD) (LOW AFFINITY |
| | | NEUROTROPHIN RECEPTOR |
| | | P75NTR). |
| 8 | NM_016192 | TRANSMEMBRANE PROTEIN WITH |
| | | EGF-LIKE AND TWO FOLLISTATIN- |
| | | LIKE DOMAINS 2; |
| | | TRANSMEMBRANE PROTEIN TENB2; |
| | | TOMOREGULIN; PUTATIVE |
| | | TRANSMEMBRANE PROTEIN WITH |
| | | EGF-LIKE AND TWO FOLLISTATIN- |
| | | LIKE DOMAINS 2. |
| 9 | NM_005221 | HOMEOBOX PROTEIN DLX-5 |
| 10 | NM_080552 | VESICULAR INHIBITORY AMINO |
| | | ACID TRANSPORTER (GABA AND |
| | | GLYCINE TRANSPORTER) |
| | | (VESICULAR GABA TRANSPORTER) |
| | | (HVIAAT) |
| 11 | NM_001208 | Transcription factor BTF3 Homolog 1 |
| 12 | NM_005904 | MOTHERS AGAINST |
| | | DECAPENTAPLEGIC HOMOLOG 7 |
| | | (SMAD 7) (MOTHERS AGAINST DPP |
| | | HOMOLOG 7) (SMAD7) (HSMAD7). |
| 13 | NM_002146 | HOMEOBOX PROTEIN HOX-B3 (HOX- |
| | | 2G) (HOX-2.7) |
| 14 | NM_025078 | Homo sapiens hypothetical protein |
| | | FLJ22378 |
| 15 | NM_001116 | ADENYLATE CYCLASE, TYPE IX (EC |
| | | 4.6.1.1) (ATP PYROPHOSPHATE- |
| | | LYASE) (ADENYLYL CYCLASE). |
| 16 | NM_001706 | B-CELL LYMPHOMA 6 PROTEIN |
| | | (BCL-6) (ZINC FINGER PROTEIN 51) |
| | | (LAZ-3 PROTEIN) (BCL-5). |
| 17 | NM_014068 | SEEK1 PROTEIN |
| 18 | NM_017745 | BCL-6 INTERACTING COREPRESSOR |
| | | ISOFORM 1 |
| 19 | NM_005643 | TRANSCRIPTION INITIATION |
| | | FACTOR TFIID 28 KDA SUBUNIT |
| | | (TAFII-28) (TAIFII28) (TFIID SUBUNIT |
| | | P30-BETA) |
| 20 | NM_007374 | HOMEOBOX PROTEIN SIX6 (SINE |
| | | OCULIS HOMEOBOX HOMOLOG 6) |
| | | (OPTIC HOMEOBOX 2) |
| | | (HOMEODOMAIN PROTEIN OPTX2). |
| 21 | AP003500. | Situated between Ensemble gene IDs |
| | 2.1.161078 | ENSESTG00002308609 and |
| | | ENSESTG00002308691 |
| 22 | AC092385. | SPIR-2 PROTEIN (FRAGMENT) |
| | 4.1.97218 | |
| 23 | AC007223. | Situated within Ensemble EST ID |
| | 5.1.159520 | ENSESTG00001971415 |
| 24 | NM_005229 | ETS-DOMAIN PROTEIN ELK-1 |
| 25 | NM_000179 | DNA MISMATCH REPAIR PROTEIN |
| | | MSH6 (MUTS-ALPHA 160 KDA |
| | | SUBUNIT) (G/T MISMATCH BINDING |
| | | PROTEIN) (GTBP) (GTMBP) (P160) |
| 26 | NM_005427 | TUMOR PROTEIN P73 (P53-LIKE |
| | | TRANSCRIPTION FACTOR) (P53- |
| | | RELATED PROTEIN). |
| 27 | NM_002093 | GLYCOGEN SYNTHASE KINASE-3 |
| | | BETA (EC 2.7.1.37) (GSK-3 BETA). |
| 28 | NM_006765 | N33 PROTEIN |
| 29 | NM_016192 | TRANSMEMBRANE PROTEIN WITH |
| | | EGF-LIKE AND TWO FOLLISTATIN- |
| | | LIKE DOMAINS 2; |
| | | TRANSMEMBRANE PROTEIN TENB2; |
| | | TOMOREGULIN; PUTATIVE |
| | | TRANSMEMBRANE PROTEIN WITH |
| | | EGF-LIKE AND TWO FOLLISTATIN- |
| | | LIKE DOMAINS 2. |
| 30 | NM_004429 | EPHRIN-B1 PRECURSOR (EPH- |
| | | RELATED RECEPTOR TYROSINE |
| | | KINASE LIGAND 2) (LERK-2) (ELK |
| | | LIGAND) (ELK-L). |
| 31 | NM_018950 | HLA-F gene for human leukocyte antigen F |
| 32 | NM_000038 | ADENOMATOUS POLYPOSIS COLI |
| | | PROTEIN (APC PROTEIN) |
| 33 | NM_000610 | CD44 ANTIGEN PRECURSOR |
| | | (PHAGOCYTIC GLYCOPROTEIN I) |
| | | (PGP-1) (HUTCH-I) (EXTRACELLULAR |
| | | MATRIX RECEPTOR-III) (ECMR-III) |
| | | (GP90 LYMPHOCYTE |
| | | HOMING/ADHESION RECEPTOR) |
| | | (HERMES ANTIGEN) |
| | | (HYALURONATE RECEPTOR) |
| | | (HEPARAN SULFATE |
| | | PROTEOGLYCAN) (EPICAN) (CDW44) |
| 34 | NM_004385 | VERSICAN CORE PROTEIN |
| | | PRECURSOR (LARGE FIBROBLAST |
| | | PROTEOGLYCAN) (CHONDROITIN |
| | | SULFATE PROTEOGLYCAN CORE |
| | | PROTEIN 2) (PG-M) (GLIAL |
| | | HYALURONATE-BINDING PROTEIN) |
| | | (GHAP) |
| 35 | | EYA 4 EnsEMBL ID ENSG00000112319 |
| 36 | NM_000455 | SERINE/THREONINE-PROTEIN |
| | | KINASE 11 (EC 2.7.1.—) |
| | | (SERINE/THREONINE-PROTEIN |
| | | KINASE LKB1) |
| 37 | NM_003242 | Transforming growth factor, beta receptor |
| | | II (TGFBR2) |
| 38 | NM_001753 | CAVEOLIN-1; CAV 1 |
| 39 | NM_001257 | CADHERIN-13 PRECURSOR |
| | | (TRUNCATED-CADHERIN) (T- |
| | | CADHERIN) (T-CAD) (HEART- |
| | | CADHERIN) (H-CADHERIN) (P105) |
| 40 | NM_000044 | ANDROGEN RECEPTOR |
| | | (DIHYDROTESTOSTERONE |
| | | RECEPTOR) |
| 41 | NM_032546 | RING FINGER PROTEIN 30 |
| 42 | NM_033178 | DOUBLE HOMEOBOX, 4; DOUBLE |
| | | HOMEOBOX PROTEIN 4. |
| 43 | NM_003573 | LATENT TRANSFORMING GROWTH |
| | | FACTOR BETA BINDING PROTEIN 4.; |
| | | LTBP4 |
| 44 | | CGI-20 PROTEIN |
| 45 | NM_014459 | PROTOCADHERIN 17; |
| | | PROTOCADHERIN 68.; PCDH 17 |
| 46 | NM_005285 | PROBABLE G PROTEIN-COUPLED |
| | | RECEPTOR GPR7.; GPR7 |
| 47 | NM_016269 | LYMPHOID ENHANCER BINDING |
| | | FACTOR 1 (LEF-1) (T CELL-SPECIFIC |
| | | TRANSCRIPTION FACTOR 1-ALPHA) |
| | | (TCF1-ALPHA) |
| 48 | AC139426. | Situated between EnsEMBL ID |
| | 2.1.188448 | ENSESTG00003302072 and EnsEMBL ID |
| | | ENSESTG00003302068 |
| 49 | NM_005904 | MOTHERS AGAINST |
| | | DECAPENTAPLEGIC HOMOLOG 7 |
| | | (SMAD 7) (MOTHERS AGAINST DPP |
| | | HOMOLOG 7) (SMAD7) (HSMAD7). |
| 50 | AL512590. | Homo sapiens mRNA for KIAA1529 protein |
| | 2.1.165432 | EnsEMBL ID ENSG00000029402 |
| 51 | NM_004852 | ONE CUT DOMAIN FAMILY MEMBER |
| | | 2 (ONECUT-2 TRANSCRIPTION |
| | | FACTOR) (OC-2). |
| 52 | NM_001900 | CYSTATIN D PRECURSOR |
| 53 | NM_001990 | EYES ABSENT HOMOLOG 3. EYA3 |
| 54 | NM_002032 | FERRITIN HEAVY CHAIN (FERRITIN |
| | | H SUBUNIT). |
| 55 | NM_002938 | RING FINGER PROTEIN 4.; RNF4 |
| 56 | | HPP1 EnsEMBL ID ENSG00000144339 |
| 57 | NM_000852 | GLUTATHIONE S-TRANSFERASE P |
| | | (EC 2.5.1.18) (GST CLASS-PI) (GSTP1- |
| | | 1) |
| 58 | NM_007084 | TRANSCRIPTION FACTOR SOX-21 |

The database contains furthermore characteristic methylation patterns of the following tissue types: Colorectal cancer (and subtypes thereof); Normal colorectal tissue; Non-colorectal carcinomas; Peripheral blood lymphocytes (and subtypes thereof); Normal tissues of non-colorectal origin; Colon polyps, and Colon inflammatory diseases.

A “subtype” of a disease in the context of the present invention refers to a distinct disease phenotype within a general group of diseases. One example would be a streptococcal infection as a subtype of a bacterial infection or an infection with Streptococcus pyogenes as a subtype of Streptococcus spec. infection. In addition, a particular strain of Streptococcus pyogenes would cause a subtype of Streptococcus pyogenes infection. Similar subtypes and/or classifications can be found, for example, in the WHO classification, such as, for example, for testicular tumors (see, for example, Mikuz G. WHO classification of testicular tumors Verh Dtsch Ges Pathol. 2002;86:67-75) or leukemia (see, for example, Todd W M. Acute myeloid leukemia and related conditions Hematol Oncol Clin North Am. 2002 April;16(2):301-19) and many other conditions and diseases, as will be apparent to the person of skill in the art. Particular subtypes are tumor types. Further examples are colorectal cancer versus colon inflammatory disease.

The database thereby provides a suitable basis for the methylation based diagnosis of a patient with a suspected colon cell proliferative disorder. A sample of the patient's DNA extracted from blood serum is bisulfite treated and the methylation pattern of the above mentioned genes is determined from the sequence of the bisulfite treated DNA.

Then, a second knowledge base is generated that comprises a plurality of expert rules for a) evaluating and b) selecting a type of disease or medical condition, wherein said evaluation and selection is based on the methylation status at selected sites of the DNA of a patient that is under analysis. That is, the second knowledge base contains rules that allow to “fit” the methylation statuses of the patient under analysis to the already present methylation patterns in the first knowledge base, whereby the expert rules provide rules that usually calculate the statistical “best fit” of the methylation pattern of the DNA of the patient under analysis with the data of the first knowledge base.

Thereafter, the result of said comparison is given out as a ranked listing, wherein the ranks reflect the statistical quality of the matches of the methylation pattern of the patient under analysis with the methylation patterns as stored in the first knowledge base, based on said expert rules of the second knowledge base. Said ranked listing will usually present the best fit on the first position of said listing that is given out, whilst at the same time the disease that is connected with said methylation pattern in the first database is displayed as well. Then, the second best fit of said comparison will be displayed at position two of said listing, whereby a ranked listing of methylation pattern fits between the first knowledge base and the methylation pattern of the patient under analysis, again based on the expert rules in the second knowledge base is generated. This procedure can be repeated in order to generate a longer ranked list of diseases. If the methylation pattern of the patient under analysis will not fit to any of the methylation patterns that are present in the first knowledge base, this result will be displayed as well.

After a listing or ranked listing of the diseases or medical conditions based on the information about the methylation status at selected sites of the DNA of the patient based on the first knowledge base and on the second knowledge base has been generated in said computing device, said ranked listing can be displayed or used for a further analysis, wherein said ranked listing is compared with a third knowledge base. Said third knowledge base comprises a plurality of different therapeutic regimens for diseases or medical conditions. Again, this third knowledge base has been generated based on data of treatment regimens that have been used for the treatment of, e.g., cancerous diseases and/or subtypes of those cancerous diseases as well. As an example, chemotherapeutical approaches that have been used for different sub-types of colorectal cancer are added into the third knowledge base, optionally together with additional information regarding the outcome of said treatment and/or adverse effects thereof. These regimens of treatments can now be compared to the ranked listing of diseases or medical conditions based on the information about the methylation status at selected sites of the DNA of the patient under analysis, by comparing said third knowledge base using expert rules that are present in a fourth knowledge base. Similar to the expert rules in the second knowledge base, these expert rules in the fourth knowledge base will now fit (match) the treatment regimen as stored in the third knowledge base to the ranked listing of diseases in the ranked listing. Again, this comparison will be put out in a ranked listing of available therapeutic treatment regimens for said patient, wherein said information that has been generated can again be displayed as a statistically ranked listing, similar to the above or stored in the computing device.

The attending physician will now be able to link the result of the methylation analysis of the DNA of the patient under analysis to a) a specific disease, in particular a subtype of an otherwise only generally diagnosed disease, and, at the same time, be able to provide the patient with a selective therapeutic regimen that is based on the specific analysis of the methylation pattern of the patient. A particular advantage of the system according to the present invention is the fact that the therapeutic regimen that is displayed will allow a much more precise analysis of the specific disease that the patient under analysis is suffering from and, at the same time, provide up-to-date therapeutic information regarding the available therapeutic treatment regimens for said specific disease. In addition, the system also allows for preventive therapeutic treatment regimens, since the statistical ranking of the methylation patterns of the diseases do not require a simple “plus/minus” analysis as currently required for common approaches.

In addition to the above knowledge bases, similar knowledge bases can be generated that contain advisory information (fifth knowledge base) that is useful for the treatment of the patient with different constituents, or other patient information in addition to the methylation status at selected sites of the DNA of the patient under analysis, such as gender, age, weight, hemoglobin information, neuropathy information, neutrophil information, pancreatitis, hepatic function, renal function, drug allergy and intolerance information.

### EXAMPLE 2

Method Employing Treatment Regimen in Order to Treat an Acute Outbreak of a Disease

A tissue sample from a patient suffering from a completely unknown or insufficiently specified acute disease is taken in the practice of a medical doctor or from medical personnel in a hospital. In the context of the present invention, the term “insufficiently specified acute disease” designates a generally diagnosed disease like, for example, cancer without specifying the exact type of cancer the patient is affected with. Further examples would be an acute viral infection or a generally specified bacterial infection. The sample of the patient contains DNA from the cells of the patient to be examined. Basically all types of samples that contain DNA from the patient can be employed in the method of the present invention. The sample can contain either specific tissue, like single types of blood cells, single types of liver cells or cells of a single tumour, or unspecific tissue, like skin, brain or other organs. The sample is then shipped together with additional patient information to a central laboratory in order to analyse the methylation statuses at selected sites of the patients' DNA. Optionally, the sample can be analysed for its methylation statuses at selected sites of the patients' DNA in a device comprising two different components as described above, which is either located in the practice of the medical doctor or, for example, in the central laboratory of a hospital. The information on the methylation pattern of the individual patient is then provided to a computing device again either located in the practice of the medical doctor or the hospital or at the central laboratory. Optionally, this information can be provided either to a remote server or from the server to the local client for further use and analyses.

In another method of the invention, tissue samples can be taken from a selected group of patients in order to, for example, monitor the outbreak of a plague caused by a specific organism or virus, like a bacterium, such as Neisseria or highly infectious viral disease.

The information about the methylation pattern(s) of the patient(s) is then processed in the computing device by an inference engine employing the information from at least two of blocks 23-27 (FIG. 2), which generates the listing of either precisely defined diseases of the individual patient or of available treatments and the corresponding individual advisory information from the information provided by the blocks.

The patient is then individually therapeutically treated based on the basis of the advisory information generated and displayed by the above-mentioned device. The treatment employs an individual treatment regimen which is specific for the type of disease the patient suffers from, together with additional individual treatment modifications which correspond to the individual needs and problems occurring with the medication of the patient, like incompatibility with a specific type of drug or active compound of a medicament as part of the treatment which is employed.

### EXAMPLE 3

Example Employing a Preventive Treatment Regimen

A tissue sample from a healthy person or a person suffering from a yet unidentified or non-acute infection or other disease like cancer in a very early stage is taken in the practice of the responsible medical doctor or from other personal in a hospital. The sample contains DNA from the patient to be examined. Therefore, the sample can contain either specific tissue, like single types of blood cells, single types of liver cells or cells of a single tumour, or unspecific tissue, like skin, brain or other organs. The sample is then shipped together with additional patient information to a central laboratory in order to analyse the methylation statuses at selected sites of the patients' DNA. Optionally, the sample can be analysed for its methylation statuses at selected sites of the patients' DNA in an analytical device as described above, having two components as described above, which is either located in the practice of the medical doctor or in the laboratory of a hospital. The information on the methylation is then provided to a computing device either located in the practice of the medical doctor or the hospital or at the central laboratory. Optionally, this information can be provided either to a remote server or from the server to the local client for further use and analyses.

The information about the methylation pattern(s) of the patient is then processed in the computing device by an inference engine employing the information from at least two of blocks 23-27 (FIG. 2), which generates the listing of either yet not identified precisely defined diseases of the individual patient or which generates a risk-assessment for the individual patient, in which the individual statistical risk for the patient is calculated, to suffer from the outbreak of an acute disease in the future. The statistical risk is calculated based on a comparison of the knowledge bases which contain the information about the methylation status of the DNA of healthy cells with the information about the methylation of the patients' DNA. A conclusion is drawn on the basis of differences found between these two methylation patterns. Further factors which can influence the outcome of such statistical diagnosis include, for example, patient information on earlier treatment regimens or acute medication.

The patient is then individually therapeutically treated based on the advisory information generated and displayed by the above-mentioned device. The preventive treatment employs an individual treatment regimen which is specific for the type of disease the patient will most likely suffer from in order to either prevent an acute outbreak of a disease and/or reduce the risk of such an outbreak. One example of a preventive treatment could be a special diet in order to limit negative effects of diabetes, allergic reactions, cancer or other diseases which can be treated most efficiently at an early stage. The preventive treatment is employed together with additional individual treatment modifications which correspond to the individual needs and problems occurring with the medication of the patient, like intolerances or allergies.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. Therefore, it is to be understood that the fore-going is illustrative of the present invention and is not to be construed as limited to the specific embodiments disclosed, and that modifications to the disclosed embodiments, as well as other embodiments, are intended to be included within the scope of the appended claims. The invention is defined by the following claims, with equivalents of the claims to be included therein.

### EXAMPLE 4

Example Employing Preventive Treatment of Progression of Disease

The following example illustrates the utility of a methylation based system for the sub classification of breast cancers. In the following example, the system according to the invention is utilized in two different ways, namely
i) for the selection of an adjuvant therapy for the treatment of a breast cancer patient subsequent to surgical resection of a primary tumour, and/or
ii) for determination of response to a treatment targeting the estrogen receptor pathways for the treatment of metastatic breast cancer.

The prediction of a response to such treatment in each setting is traditionally based on the patient, disease and tumor characteristics e.g. age and estrogen receptor status.

First Knowledge Base

The first knowledge base consists of methylation profiles of known disease/medical condition status, in this case the prognosis for response to treatment of breast cancer patients with therapies targeting the estrogen receptor pathways. For the initial build-up of the database system for use in the method of the present invention, a first knowledge base is generated by collecting information regarding the methylation status of selected sites of DNA of a large number of individuals, wherein characteristic methylation profiles of responders and non-responders to therapies targetting the estrogen receptor pathway (in both adjuvant and metastatic settings) are stored.

In the following example, the CpG methylation levels of the following genes or their regulatory regions (‘methylation profiles’) characteristic of responders and non-responders are stored in the form of a database: Hypermethylation of the genes STMN1, SFN, S100A2, TGFBR2, TP53, PTGS2, FGFR1, SYK, PITX2, GRIN2D, PSA, CGA, CYP2D6, MSMB, COX7A2L, VTN, PRKCD, ONECUT2, WBP11, CYP2D6, DAG1, ERBB2, S100A2, TFF1, TP53, TMEFF2, ESR1, SYK, RASSF1, PITX2, PSAT1, CGA and PCAF.

Second Knowledge Base

The second knowledge base consists of expert rules for the analysis of patient (‘test’) methylation profiles as compared to the methylation profiles of this stored in knowledge base 1, in this case the prognosis for a response to treatment of breast cancer patients with therapies targeting the estrogen receptor pathways. This consists of two steps:

Hypothesis Testing

The main task is to identify markers that show significant differences in the average degree of methylation between two classes (responders and non-responders to treatment). A significant difference is detected when the null-hypothesis that the average methylation of the two classes is identical can be rejected with p<0.05. Because this test is applied to a whole set of potential markers the p-values for multiple testing have to be corrected. This is done by applying the False Discovery Rate (FDR) method (Dudoit et al., 2002).

For testing the null hypothesis that the methylation levels in the two classes are identical the likelihood ratio test for logistic regression models (Venables and Ripley, 2002) is used. The logistic regression model for a single marker is a linear combination of methylation measurements from all CpG positions in the respective genomic region of interest (ROI). A significant p-value for a marker means that this ROI has some systematic correlation to the question of interest as given by the two classes.

Class Prediction by Supervised Learning

In order to give a reliable estimate of how well the CpG ensemble of a selected marker can differentiate between different tissue classes (e.g. responders and non-responders) we can its prediction accuracy is determined by classification. For that purpose a methylation profile based prediction function using a certain set of tissue samples with their class label is calculated. This step is called training and it exploits the prior knowledge represented by the data labels. The prediction accuracy of that function is then tested by cross-validation or on a set of independent samples. As a method of choice, the support vector machine (SVM) algorithm (Duda (2001), Christiannini (2000)) is used to learn the prediction function. If not stated otherwise, for this report the risk associated with false positive or false negative classifications are set to be equal relative to the respective class sizes. It follows that the learning algorithm obtains a class prediction function with the objective to optimize accuracy on an independent test sample set. Therefore sensitivity and specificity of the resulting classifier can be expected to be approximately equal.

As a result, the hypomethylation of the genes stathmin 1/oncoprotein 18 (STMN1) and stratifin/14-3-3 protein a (SFN) and a hypermethylation of all other genes in the set were characteristics of responders.

Patient 1: Adjuvant Setting

In a patient presented with primary breast cancer tumor, surgical resection was carried out. DNA was isolated from a sample of the resected breast tumor; and the methylation status of the genes ESR1, PCAF, PITX2, TMEFF2, WBP11 and ERBB2 was determined by means of LightCycler based PCR assays conducted in the presence of methylation specific probes. All genes were hypermethylated. By use of expert rules (algorithms generated according to knowledge base 2) the methylation profile of the patient was compared to that of knowledge base 1. The patient was thereby classified as breast cancer patient responder to estrogen pathway targeting therapy.

Patient 2: Metastatic Setting

The patient was presented with recurrent metastatic breast cancer. DNA was isolated from a sample of breast tumor tissue preserved from prior surgery. The methylation status of the genes PSA-T, STMN1, GRIN2D, TGFBR2 and S100A2 was determined by means of LightCycler based PCR assays conducted in the presence of methylation specific probes. The patient was classified as breast cancer patient non-responder to estrogen pathway targeting therapy

Ranking of Therapies

The patient characteristics were then used in the ranking of therapies contained in a third knowledge base. The following patient characteristics (where applicable) were used:
Age
Tumor Size
Nodal status
Hormone receptor status (including estrogen and progesterone receptors)
Stage
Prior treatment
Previous cancer
Menopausal stage
Responder status to estrogen pathway targeting therapies
Lymph node status
Molecular markers (including HER2 expression)

The third knowledge base includes all recommended breast cancer treatments including radiation treatment, ovarian ablation, other surgery, anthracyclines, taxanes, alkylating agents, Progestins, SERDs (Selective Estrogen Receptor Downregulators), Aromatase inhibitors, SERMs (Selective Estrogen-Reuptake Inhibitors), Aromatase inhibitors, biological response modifiers and other hormonal therapies.

These include:
Adriamycin (doxorubicin); Aredia (generic name, pamidronate disodium) Arimidex (anastrozole); Aromasin (exemestane);
   Chemotherapy Regimens
Cytoxan (cyclophosphamide); Ellence (epirubicin); Fareston (toremifene); Femara (letrozole); Herceptin (trastuzumab); Megace (megestrol); Tamoxifen (Nolvadex); Taxol (paclitaxel); Taxotere (docetaxel); Xeloda (capecitabine); Zoladex (goserelin acetate)
   Chemotherapy combined regimens
cyclophosphamide (Cytoxan), methotrexate (Amethopterin, Mexate, Folex), and fluorouracil (Fluorouracil, 5-Fu, Adrucil) (this therapy is called CMF); cyclophosphamide, doxorubicin (Adriamycin), and fluorouracil (this therapy is called CAF); doxorubicin (Adriamycin) and cyclophosphamide (this therapy is called AC); doxorubicin (Adriamycin) and cyclophosphamide with paclitaxel (Taxol); doxorubicin (Adriamycin), followed by CMF; cyclophosphamide, epirubicin (Ellence), and fluorouracil; docetaxel, doxorubicin, and cyclophosphamide (TAC); gemcitabine and trastuzumab; vinorelbine and trastuzumab.

The recommended ranked therapies are as follows.

Patient 1:
1. Tamoxifen treatment,
2. Doxorubicin (Adriamycin) and cyclophosphamide combined therapy, and
3. Cyclophosphamide, doxorubicin (Adriamycin), fluorouracil combined therapy

Patient 2:
1. Gemcitabine and trastuzumab combined therapy,
2. Vinorelbine and trastuzumab combined therapy, and
3. Docetaxel, doxorubicin, and cyclophosphamide (TAC)

In the following, the patients were treated accordingly using a regular treatment scheme.

1. A method for guiding the selection of a therapeutic treatment regimen for a patient with a disease or medical condition, said method comprising:
(A) providing information about the methylation status at selected sites of the DNA of the patient to a computing device comprising:
i. a first knowledge base comprising information about a plurality of different methylation statuses at selected sites of the DNA in cells with a known disease or class thereof, medical condition and/or healthy cells,
ii. a second knowledge base comprising a plurality of expert rules for evaluating and selecting a type of disease or medical condition based on the methylation status at selected sites of the DNA of a patient,
(B) generating in said computing device a listing or ranked listing of diseases or medical conditions based on the information about the methylation status at selected sites of the DNA of the patient, the first knowledge base and the second knowledge base.

2. The method according to claim 1 wherein said medical condition is differential therapeutic treatment response.

3. The method according to claim 1 wherein the class of disease is a grade, stage or molecular subclassification.

4. The method according to claim 1, further comprising:
i. a third knowledge base comprising a plurality of different therapeutic regimens for diseased cells or medical conditions,
ii. a fourth knowledge base comprising a plurality of expert rules for evaluating and selecting therapeutic treatment regimens for diseased cells or medical conditions, and
(C) generating in said computing device a ranked listing of available therapeutic treatment regimens for said patient based on the information generated in step (B) and the third knowledge base and fourth knowledge base.

5. The method according to claim 4, characterized in that the therapeutic regimen is a preventive therapeutic treatment regimen.

6. The method according to claim 4, further comprising:
i. a fifth knowledge base comprising advisory information useful for the treatment of a patient with different constituents of said different therapeutic treatment regimens; and
(D) generating in said computing device advisory information for one or more treatment regimens in said ranked listing based on the information generated in step (C) according to claim 2 and the fifth knowledge base.

7. The method according to claim 4, further comprising the steps of:
(E) entering a user-defined therapeutic treatment regimen for said disease or medical condition that is not included in said third knowledge base; and
(F) generating in said computing device advisory information for one or more user-defined combination therapeutic treatment regimen.

8. The method according to claim 1, in which said patient information in addition to the information about the methylation status at selected sites of the DNA comprises gender, age, weight, hemoglobin information, neuropathy information, neutrophil information, pancreatitis, hepatic function, renal function, drug allergy and intolerance information.

9. The method according to claim 1, wherein said patient information includes prior therapeutic treatment regimen information.

10. The method according to claim 1, wherein said patient information includes prior patient information stored in said computing device.

11. The method according to claim 6, said advisory information including:
i. warnings to take the patient off a contraindicated drug before initiating a corresponding therapeutic treatment regimen; and information clinically useful to implement a corresponding therapeutic treatment regimen.

12. The method according to claim 1, wherein said computing device comprises a sixth knowledge base comprising patient therapeutic treatment regimen history, said advisory information including previous therapeutic treatment regimen information extracted from said sixth knowledge base.

13. The method according to claim 1, wherein said disease or medical condition is a cardiovascular disease, a pulmonary disease, a neurologic disease, diabetes, a urinary tract infection, hepatitis, HIV infection, cancer or other cell proliferative disorders.

14. The method according to claim 1, wherein drug dosage information is recommended and adjusted if necessary depending upon said patient information.

15. The method according to claim 1, further comprising the step of:
(G) accessing, via said computing device, information for one or more therapeutic treatment regimens from a drug reference source.

16. A method for treatment of a patient with a disease or medical condition, said method comprising:
i) isolating a DNA-containing sample from said patient;
ii) analyzing cytosine methylation patterns at selected sites of the DNA contained in said sample;
iii) providing data about the methylation status at selected sites of the DNA of the patient thereby creating a first knowledge base comprising said data, a second knowledge base comprising information about a plurality of different methylation statuses at selected sites of the DNA in cells with a known disease or medical condition and/or healthy cells,
iv) a third knowledge base comprising a plurality of expert rules for evaluating and selecting a type of disease or medical condition based on the methylation status at selected sites of the DNA of a patient, and
v) generating a ranked listing of diseases or medical conditions based on the data of the first knowledge base, the second knowledge base and the third knowledge base.

17. The method according to claim 16 wherein said medical condition is differential therapeutic treatment response.

18. The method according to claim 16 wherein the class of disease is a grade, stage or molecular subclassification.

19. The method according to claim 16, in which the data is provided to a computing device.

20. The method according to claim 16, further comprising:
i) a fourth knowledge base comprising a plurality of different therapeutic regimens for diseased cells or medical conditions,
ii) a fifth knowledge base comprising a plurality of expert rules for evaluating and selecting therapeutic treatment regimens for diseased cells or medical conditions, and
iii) generating a ranked listing of available therapeutic treatment regimens for said patient based on the information generated in step (D) according to claim 14 and the fourth knowledge base and the fifth knowledge base.

21. The method according to claim 16 wherein said medical condition is differential therapeutic treatment response.

22. The method according to claim 16 wherein the class of disease is a grade, stage or molecular subclassification.

23. The method according to claim 20, characterized in that the therapeutic treatment regimen is a preventive treatment regimen.

24. The method according to claim 20, further comprising:
i) a sixth knowledge base comprising advisory information useful for the treatment of a patient with different constituents of said different therapeutic treatment regimens; and
ii) generating advisory information for one or more specific treatment regimens in said ranked listing based on the information generated in step (E) according to claim 14 and the sixth knowledge base; and
iii) providing said one or more specific treatment regimens to said patient with a disease or medical condition based on the advisory information generated in step (F).

25. The method according to claim 20, further comprising the steps of:
i) entering a user-defined therapeutic treatment regimen for said disease or medical condition that is not included in said fourth knowledge base; and
ii) generating advisory information for one or more user-defined combination therapeutic treatment regimen.

26. The method according to claim 16, in which said patient data in addition to the data about the methylation status at selected sites of the DNA comprises gender, age, weight, hemoglobin information, neuropathy information, neutrophil information, pancreatitis, hepatic function, renal function, drug allergy and intolerance information.

27. The method according to claim 16, wherein said patient data includes prior therapeutic treatment regimen information.

28. The method according to claim 16, wherein said patient data includes prior patient information stored in said computing device.

29. The method according to claim 18, said advisory information including:
warnings to take the patient off a contraindicated drug before initiating a corresponding therapeutic treatment regimen; and information clinically useful to implement a corresponding therapeutic treatment regimen.

30. The method according to claim 24, comprising a seventh knowledge base comprising patient therapeutic treatment regimen history, said advisory information including previous therapeutic treatment regimen information extracted from said seventh knowledge base.

31. The method according to claim 16, wherein said disease or medical condition is a cardiovascular disease, a pulmonary disease, a neurologic disease,diabetes, a urinary tract infection, hepatitis, HIV infection, cancer or other cell proliferative disorders.

32. The method according to claim 16, wherein drug dosage information is recommended and adjusted if necessary depending upon said patient information.

33. The method according to claim 16, further comprising the step of:
(J) accessing, via a computing device, information for one or more therapeutic treatment regimens from a drug reference source.

34. A system for guiding the selection of a therapeutic treatment regimen for a patient with a disease or medical condition, said system comprising:
(A) a computing device comprising:
i. a first knowledge base comprising information about a plurality of different methylation statuses at selected sites of the DNA in cells with a known disease or medical condition and/or healthy cells,
ii. a second knowledge base comprising a plurality of expert rules for evaluating and selecting a type of disease or medical condition based on the methylation status at selected sites of the DNA of a patient,
(B) means for providing information about the methylation status at selected sites of the DNA of the patient to computing device;
(C) means for generating in said computing device a ranked listing of diseases or medical conditions based on the information about the methylation status at selected sites of the DNA of the patient, the first knowledge base and the second knowledge base.

35. The system according to claim 34 wherein said medical condition is differential therapeutic treatment response.

36. The system according to claim 34 wherein the class of disease is a grade, stage or molecular subclassification.

37. The system according to claim 34, further comprising:
i. a third knowledge base comprising a plurality of different therapeutic regimens and/or preventive therapeutic treatment regimens for diseased cells or medical conditions,
ii. a fourth knowledge base comprising a plurality of expert rules for evaluating and selecting therapeutic treatment regimens for diseased cells or medical conditions; and
(D) means for generating in said computing device a listing or ranked listing of available therapeutic treatment regimens for said patient.

38. The system according to claim 34, further comprising:
i. a fifth knowledge base comprising advisory information useful for the treatment of a patient with different constituents of said different therapeutic treatment regimens; and
(E) means for generating in said computing device advisory information for one or more treatment regimens in said ranked listing.

39. The system according to claim 37, further comprising:
(F) means for entering a user-defined therapeutic treatment regimen for said disease or medical condition that is not included in said third knowledge base; and
(G) means for generating in said computing device advisory information for one or more user-defined combination therapeutic treatment regimen.

40. The system according to claim 34, said patient information in addition to the information about the methylation status at selected sites of the DNA comprises gender, age, weight, hemoglobin information, neuropathy information, neutrophil information, pancreatitis, hepatic function, renal function, drug allergy and intolerance information.

41. The system according to claim 34, wherein said patient information includes prior therapeutic treatment regimen information.

42. The system according to claim 34, wherein said patient information includes prior patient information stored in said computing device.

43. The system according to claim 34, said advisory information including:
warnings to take the patient off a contraindicated drug before initiating a corresponding therapeutic treatment regimen; and information clinically useful to implement a corresponding therapeutic treatment regimen.

44. The system according to claim 34, wherein said computing device comprises a sixth knowledge base comprising patient therapeutic treatment regimen history, said advisory information including previous therapeutic treatment regimen information extracted from said sixth knowledge base.

45. The system according to claim 34, wherein said disease or medical condition is a cardiovascular disease, a pulmonary disease, a neurologic disease, diabetes, a urinary tract infection, hepatitis, HIV infection, cancer or other cell proliferative disorders.

46. The system according to claim 34, wherein drug dosage information is recommended and adjusted if necessary depending upon said patient information.

47. The system according to claim 34, further comprising:
(H) means for accessing, via said computing device, information for one or more therapeutic treatment regimens from a standard drug reference source.

48. A computer program product for guiding the selection of a therapeutic treatment regimen for a patient with a disease or medical condition, said computer program product comprising
a computer usable storage medium having computer readable program code means embodied in the medium, the computer readable program code means comprising:
(A) computer readable program code means for generating:
i) a first knowledge base comprising information about a plurality of different methylation statuses at selected sites of the DNA in cells with a known disease or medical condition and/or healthy cells,
ii) a second knowledge base comprising a plurality of expert rules for evaluating and selecting a type of disease or medical condition based on the methylation status at selected sites of the DNA of a patient,
iii) a third knowledge base comprising a plurality of different therapeutic regimens and/or preventive therapeutic regimens for diseased cells or medical conditions,
iv) a fourth knowledge base comprising a plurality of expert rules for evaluating and selecting therapeutic treatment regimens for diseased cells or medical conditions
v) a fifth knowledge base comprising advisory information useful for the treatment of a patient with different constituents of said different therapeutic treatment regimens; and
(B) computer readable program code means for providing information about the methylation status at selected sites of the DNA of the patient;
(C) computer readable program code means for generating a ranked listing of diseases or medical conditions based on the information about the methylation status at selected sites of the DNA of the patient; and
(D) computer readable program code means for generating in said computing device a ranked listing of available therapeutic treatment regimens for said patient.

49. The computer program product according to claim 48 wherein said medical condition is differential therapeutic treatment response.

50. The computer program product according to claim 48 wherein the class of disease is a grade, stage or molecular subclassification.

51. The computer program product according to claim 48, further comprising:
(E) computer readable program code means for generating in said computing device advisory information for one or more treatment regimens in said ranked listing.

52. The computer program product according to claim 48, further comprising:
(F) computer readable program code means entering a user-defined therapeutic treatment regimen for said disease or medical condition that is not included in said third knowledge base;
(G) computer readable program code means for generating in said computing device advisory information for one or more user-defined combination therapeutic treatment regimen.

53. The computer program product according to claim 48, said patient information in addition to the information about the methylation status at selected sites of the DNA comprises gender, age, weight, hemoglobin information, neuropathy information, neutrophil information, pancreatitis, hepatic function, renal function, drug allergy and intolerance information.

54. The computer program product according to claim 48, said patient information including prior therapeutic treatment regimen information.

55. The computer program product according to claim 48, wherein said patient information includes prior patient information.

56. The computer program product according to claim 48, said advisory information including: warnings to take the patient off a contraindicated drug before initiating a corresponding therapeutic treatment regimen; and information clinically useful to implement a corresponding therapeutic treatment regimen.

57. The computer program product according to claim 48 wherein said computer readable program code means comprises computer readable program code means for generating a sixth knowledge base comprising patient therapeutic treatment regimen history, said advisory information including previous therapeutic treatment regimen information extracted from said sixth knowledge base.

58. The computer program product according to claim 48, wherein said disease or medical condition is a cardiovascular disease, a pulmonary disease, a neurologic disease, diabetes, a urinary tract infection, hepatitis, HIV infection, cancer or other cell proliferative disorders.

59. The computer program product according to claim 48, wherein drug dosage information is recommended and adjusted if necessary depending upon said patient information.

60. The computer program product according to claim 48, further comprising:
(H) computer readable program code means for accessing information for one or more therapeutic treatment regimens from a standard drug reference source.
